SEQUENCE LISTING (83 sequences)

SEQ ID NO: 1 - protein, 1273 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 21 aa, Artificial Sequence
  Synthetic Construct
ERDISTEIYQAGSTPCNGVEG

SEQ ID NO: 3 - protein, 18 aa, Artificial Sequence
  Synthetic Construct
IRGDEVRQIAPGQTGKIA

SEQ ID NO: 4 - protein, 21 aa, Artificial Sequence
  Synthetic Construct
QFGRDIADTTDAVRDPQTLEI

SEQ ID NO: 5 - protein, 18 aa, Artificial Sequence
  Synthetic Construct
EQDKNTQEVFAQVKQIYK

SEQ ID NO: 6 - protein, 18 aa, Artificial Sequence
  Synthetic Construct
DSFKEELDKYFKNHTSPD

SEQ ID NO: 7 - protein, 18 aa, Artificial Sequence
  Synthetic Construct
GFNCYFPLQSYGFQPTNG

SEQ ID NO: 8 - protein, 24 aa, Artificial Sequence
  Synthetic Construct
KLNDLCFTNVYADSFVIRGDEVRQ

SEQ ID NO: 9 - protein, 19 aa, Artificial Sequence
  Synthetic Construct
GAISLAPKAQIKESLRAEL

SEQ ID NO: 10 - protein, 18 aa, Artificial Sequence
  Synthetic Construct
VTSEHELTCQAEGYPKAE

SEQ ID NO: 11 - protein, 18 aa, Artificial Sequence
  Synthetic Construct
KLLSLIKGVIVHRLEGVE

SEQ ID NO: 12 - protein, 21 aa, Artificial Sequence
  Synthetic Construct
FNNFTVSFWLRVPKVSASHLE

SEQ ID NO: 13 - protein, 4 aa, Artificial Sequence
  Synthetic Construct
GPSL

SEQ ID NO: 14 - protein, 46 aa, Artificial Sequence
  Synthetic Construct
FNNFTVSFWLRVPKVSASHLEGPSLERDISTEIYQAGSTPCNGVEG

SEQ ID NO: 15 - protein, 43 aa, Artificial Sequence
  Synthetic Construct
KLLSLIKGVIVHRLEGVEGPSLERDISTEIYQAGSTPCNGVEG

SEQ ID NO: 16 - protein, 43 aa, Artificial Sequence
  Synthetic Construct
FNNFTVSFWLRVPKVSASHLEGPSLIRGDEVRQIAPGQTGKIA

SEQ ID NO: 17 - protein, 46 aa, Artificial Sequence
  Synthetic Construct
FNNFTVSFWLRVPKVSASHLEGPSLQFGRDIADTTDAVRDPQTLEI

SEQ ID NO: 18 - protein, 43 aa, Artificial Sequence
  Synthetic Construct
FNNFTVSFWLRVPKVSASHLEGPSLEQDKNTQEVFAQVKQIYK

SEQ ID NO: 19 - protein, 43 aa, Artificial Sequence
  Synthetic Construct
FNNFTVSFWLRVPKVSASHLEGPSLDSFKEELDKYFKNHTSPD

SEQ ID NO: 20 - protein, 43 aa, Artificial Sequence
  Synthetic Construct
FNNFTVSFWLRVPKVSASHLEGPSLGFNCYFPLQSYGFQPTNG

SEQ ID NO: 21 - protein, 49 aa, Artificial Sequence
  Synthetic Construct
FNNFTVSFWLRVPKVSASHLEGPSLKLNDLCFTNVYADSFVIRGDEVRQ

SEQ ID NO: 22 - protein, 44 aa, Artificial Sequence
  Synthetic Construct
FNNFTVSFWLRVPKVSASHLEGPSLGAISLAPKAQIKESLRAEL

SEQ ID NO: 23 - protein, 43 aa, Artificial Sequence
  Synthetic Construct
FNNFTVSFWLRVPKVSASHLEGPSLVTSEHELTCQAEGYPKAE

SEQ ID NO: 24 - protein, 21 aa, Artificial Sequence
  Synthetic Construct
GEVGNCPTSGAQYIETSIDRE

SEQ ID NO: 25 - protein, 18 aa, Artificial Sequence
  Synthetic Construct
AIKGTQGPAIQRVEDGRI

SEQ ID NO: 26 - protein, 21 aa, Artificial Sequence
  Synthetic Construct
IELTQPDRVADTTDAIDRGFQ

SEQ ID NO: 27 - protein, 18 aa, Artificial Sequence
  Synthetic Construct
KYIQKVQAFVEQTNKDQE

SEQ ID NO: 28 - protein, 18 aa, Artificial Sequence
  Synthetic Construct
DPSTHNKFYKDLEEKFSD

SEQ ID NO: 29 - protein, 18 aa, Artificial Sequence
  Synthetic Construct
GNTPQFGYSQLPFYCNFG

SEQ ID NO: 30 - protein, 24 aa, Artificial Sequence
  Synthetic Construct
QRVEDGRIVFSDAYVNTFCLDNLK

SEQ ID NO: 31 - protein, 46 aa, Artificial Sequence
  Synthetic Construct
FNNFTVSFWLRVPKVSASHLEGPSLGEVGNCPTSGAQYIETSIDRE

SEQ ID NO: 32 - protein, 43 aa, Artificial Sequence
  Synthetic Construct
FNNFTVSFWLRVPKVSASHLEGPSLAIKGTQGPAIQRVEDGRI

SEQ ID NO: 33 - protein, 46 aa, Artificial Sequence
  Synthetic Construct
FNNFTVSFWLRVPKVSASHLEGPSLIELTQPDRVADTTDAIDRGFQ

SEQ ID NO: 34 - protein, 43 aa, Artificial Sequence
  Synthetic Construct
FNNFTVSFWLRVPKVSASHLEGPSLKYIQKVQAFVEQTNKDQE

SEQ ID NO: 35 - protein, 43 aa, Artificial Sequence
  Synthetic Construct
FNNFTVSFWLRVPKVSASHLEGPSLDPSTHNKFYKDLEEKFSD

SEQ ID NO: 36 - protein, 43 aa, Artificial Sequence
  Synthetic Construct
FNNFTVSFWLRVPKVSASHLEGPSLGNTPQFGYSQLPFYCNFG

SEQ ID NO: 37 - protein, 49 aa, Artificial Sequence
  Synthetic Construct
FNNFTVSFWLRVPKVSASHLEGPSLQRVEDGRIVFSDAYVNTFCLDNLK

SEQ ID NO: 38 - protein, 21 aa, Artificial Sequence
  Synthetic Construct
ERDISTEIYQAGNTPCNGVEG

SEQ ID NO: 39 - protein, 21 aa, Artificial Sequence
  Synthetic Construct
ERDISTEIYQAGNTPCNGVKG

SEQ ID NO: 40 - protein, 21 aa, Artificial Sequence
  Synthetic Construct
ERDISTEIYQAGNTPCNGVQG

SEQ ID NO: 41 - protein, 21 aa, Artificial Sequence
  Synthetic Construct
ERDISTEIYQAGNTPCNGVQR

SEQ ID NO: 42 - protein, 18 aa, Artificial Sequence
  Synthetic Construct
IRGDEVRQIAPGQTGNIA

SEQ ID NO: 43 - protein, 18 aa, Artificial Sequence
  Synthetic Construct
GLNCYFPLQSYGFQPTNG

SEQ ID NO: 44 - protein, 18 aa, Artificial Sequence
  Synthetic Construct
GFNCYSPLQSYGFQPTNG

SEQ ID NO: 45 - protein, 18 aa, Artificial Sequence
  Synthetic Construct
GFNCYFPLQPYGFQPTNG

SEQ ID NO: 46 - protein, 18 aa, Artificial Sequence
  Synthetic Construct
GFNCYFPLQSYGFQPTYG

SEQ ID NO: 47 - protein, 18 aa, Artificial Sequence
  Synthetic Construct
RFNCYFPLQSYGFQPTNG

SEQ ID NO: 48 - protein, 46 aa, Artificial Sequence
  Synthetic Construct
FNNFTVSFWLRVPKVSASHLEGPSLERDISTEIYQAGNTPCNGVEG

SEQ ID NO: 49 - protein, 46 aa, Artificial Sequence
  Synthetic Construct
FNNFTVSFWLRVPKVSASHLEGPSLERDISTEIYQAGNTPCNGVKG

SEQ ID NO: 50 - protein, 46 aa, Artificial Sequence
  Synthetic Construct
FNNFTVSFWLRVPKVSASHLEGPSLERDISTEIYQAGNTPCNGVQG

SEQ ID NO: 51 - protein, 46 aa, Artificial Sequence
  Synthetic Construct
FNNFTVSFWLRVPKVSASHLEGPSLERDISTEIYQAGNTPCNGVQR

SEQ ID NO: 52 - protein, 43 aa, Artificial Sequence
  Synthetic Construct
FNNFTVSFWLRVPKVSASHLEGPSLIRGDEVRQIAPGQTGNIA

SEQ ID NO: 53 - protein, 43 aa, Artificial Sequence
  Synthetic Construct
FNNFTVSFWLRVPKVSASHLEGPSLGLNCYFPLQSYGFQPTNG

SEQ ID NO: 54 - protein, 43 aa, Artificial Sequence
  Synthetic Construct
FNNFTVSFWLRVPKVSASHLEGPSLGFNCYSPLQSYGFQPTNG

SEQ ID NO: 55 - protein, 43 aa, Artificial Sequence
  Synthetic Construct
FNNFTVSFWLRVPKVSASHLEGPSLGFNCYFPLQPYGFQPTNG

SEQ ID NO: 56 - protein, 43 aa, Artificial Sequence
  Synthetic Construct
FNNFTVSFWLRVPKVSASHLEGPSLGFNCYFPLQSYGFQPTYG

SEQ ID NO: 57 - protein, 43 aa, Artificial Sequence
  Synthetic Construct
FNNFTVSFWLRVPKVSASHLEGPSLRFNCYFPLQSYGFQPTNG

SEQ ID NO: 58 - protein, 20 aa, Artificial Sequence
  Synthetic Construct
CTCCCAACCCCAGAGGTAGT

SEQ ID NO: 59 - protein, 20 aa, Artificial Sequence
  Synthetic Construct
AGACCCCAGATCCAGAAAGG

SEQ ID NO: 60 - protein, 20 aa, Artificial Sequence
  Synthetic Construct
CCTGGCTGAAAGACCAGAAC

SEQ ID NO: 61 - protein, 20 aa, Artificial Sequence
  Synthetic Construct
TCAAATTAGCCACTCGCACA

SEQ ID NO: 62 - protein, 19 aa, Artificial Sequence
  Synthetic Construct
NSVDDALINSTIYSYFPSV

SEQ ID NO: 63 - protein, 17 aa, Artificial Sequence
  Synthetic Construct
PGINGKAIHLVNNQSSE

SEQ ID NO: 64 - protein, 15 aa, Artificial Sequence
  Synthetic Construct
QYIKANSKFIGITEL

SEQ ID NO: 65 - protein, 15 aa, Artificial Sequence
  Synthetic Construct
LSEIKGVIVHRLEGV

SEQ ID NO: 66 - protein, 15 aa, Artificial Sequence
  Synthetic Construct
FFLLTRILTIPQSLN

SEQ ID NO: 67 - protein, 20 aa, Artificial Sequence
  Synthetic Construct
TCGVGVRVRSRVNAANKKPE

SEQ ID NO: 68 - protein, 1255 aa, Artificial Sequence
  Synthetic Construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 69 - protein, 1353 aa, Artificial Sequence
  Synthetic Construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 70 - protein, 20 aa, Artificial Sequence
  Synthetic Construct
ERDISNVPFSPDGKPCTPPA

SEQ ID NO: 71 - protein, 20 aa, Artificial Sequence
  Synthetic Construct
TEVPQLVNANQYSPCVSIVP

SEQ ID NO: 72 - protein, 18 aa, Artificial Sequence
  Synthetic Construct
VKGDDVRQIAPGQTGVIA

SEQ ID NO: 73 - protein, 18 aa, Artificial Sequence
  Synthetic Construct
YPLSMKSDLSVSSAGPIS

SEQ ID NO: 74 - protein, 21 aa, Artificial Sequence
  Synthetic Construct
QFGRDVSDFTDSVRDPKTSEI

SEQ ID NO: 75 - protein, 20 aa, Artificial Sequence
  Synthetic Construct
RFVYDAYQNLVGYYSDDGNY

SEQ ID NO: 76 - protein, 18 aa, Artificial Sequence
  Synthetic Construct
EQDRNTREVFAQVKQMYK

SEQ ID NO: 77 - protein, 18 aa, Artificial Sequence
  Synthetic Construct
RQDDSVRNLFASVKSSQS

SEQ ID NO: 78 - protein, 18 aa, Artificial Sequence
  Synthetic Construct
DSFKEELDKYFKNHTSPD

SEQ ID NO: 79 - protein, 18 aa, Artificial Sequence
  Synthetic Construct
IDFQDELDEFFKNVSTSI

SEQ ID NO: 80 - protein, 18 aa, Artificial Sequence
  Synthetic Construct
ALNCYWPLNDYGFYTTTG

SEQ ID NO: 81 - protein, 28 aa, Artificial Sequence
  Synthetic Construct
PSTVWEDGDYYRKQLSPLEGGGWLVASG

SEQ ID NO: 82 - protein, 24 aa, Artificial Sequence
  Synthetic Construct
YNSTFFSTFKCYGVSATKLNDLCF

SEQ ID NO: 83 - protein, 24 aa, Artificial Sequence
  Synthetic Construct
LSLFSVNDFTCSQISPAAIASNCY

(19) United States
(12) Patent Application Publication
(10) Pub. No.: US 2024/0226279 A1
(43) Pub. Date: Jul. 11, 2024

(54) HUMAN ANTI-CORONAVIRUS PEPTIDE VACCINES AND METHODS OF THEIR USE

(71) Applicant: Ohio State Innovation Foundation, Columbus, OH (US)

(72) Inventor: Pravin T.P. Kaumaya, Westerville, OH (US)

(21) Appl. No.: 18/558,973

(22) PCT Filed: May 3, 2022
(86) PCT No.: PCT/US2022/027482

§ 371 Date: Nov. 3, 2023

Related U.S. Application Data
(60) Provisional application No. 63/183,222, filed on May 3, 2021.

(51) Int. Cl. A61K 39/215; A61P 31/14; A61P 37/04

(52) U.S. Cl. CPC A61K 39/215; A61P 31/14; A61P 37/04

(57) ABSTRACT

Disclosed are compositions related to modified coronavirus S protein peptides, synthetic coronavirus S protein peptides, chimeric coronavirus S protein peptides, anti-coronavirus S protein antibodies and methods of treating coronavirus infections using said peptides or antibodies.

### I. CROSS REFERENCE TO RELATED APPLICATIONS

This application claims the benefit of U.S. Provisional Application No. 63/183,222, filed on May 3, 2021, which is incorporated herein by reference in its entirety.

### II. BACKGROUND

The 2019 novel coronavirus (2019-nCoV) has caused over 412 million confirmed cases globally, including 5.8 million deaths as of February 2022. In the USA 77 million infected people have been identified with 920,000 deaths. According to the Washington Post Covid-19 is on pace to be the largest single killer of Americans (Apr. 16, 2020). As of 8 Apr. 2020, the global COVID-19 vaccine R&D landscape includes 115 vaccine candidates of which 78 are confirmed as active exploratory or preclinical stages.

The pandemic caused by coronavirus SARS-CoV-2 presents a serious global public health emergency requiring urgent need of prophylactic and therapeutic vaccines. Currently, however, there is limited specific antiviral treatment or 3 vaccines licensed under EUA for this disease and there is an urgent need to rapidly develop new agents. Even though remdesivir and some other medications gave COVID-19 patients some help, but the vaccine against SARS-CoV2 human coronavirus were eagerly to be developed and end the pandemic. Several emergency authorized vaccines have been injected to protect millions of people worldwide. They are either made from SARS-CoV2 viral RNA or heat inactivated virus etc. Here, we introduce novel peptide based SARS-CoV2 vaccines which were designed from SARS-CoV2 spike protein. These vaccines can directly boost immune system instead of translate viral RNA into protein to build a defense response.

Unlike vaccines being developed by Big Pharma or biotech and academic institutions in the USA and around the world, we have a unique validated vaccine approach that is based on chimeric B-cell epitope peptides incorporating a “promiscuous” T cell epitope that elicit natural high affinity polyclonal antibodies. In general, synthetic peptides are safe, easily synthesized and characterized, are cost effective and lack of toxicity. There are several advantages of peptide vaccines (B- or T-epitope based vaccines) and therapeutics over other vaccine approaches. Active vaccination offers many advantages, including the activation of immune responses against viral antigens. Additional advantages of chimeric B & T-cell vaccines are exquisite specificity and the potential for a durable treatment effect that can be recalled due to immunologic memory. What are needed are novel, unique and safe vaccines that rapidly induce potent long-lasting virus-specific immune responses against coronavirus spike (S) envelope protein including the receptor bonding domain (RBD).

### III. SUMMARY

Disclosed are methods and compositions related to novel synthetic coronavirus spike protein peptides, chimeric coronavirus spike protein peptides, and modified coronavirus spike protein peptides.

In one aspect, disclosed herein are chimeric peptides for stimulating an immune response to a coronavirus spike protein comprising one or more SARS-CoV-2 B cell epitopes, a T helper (Th) epitope (such as, for example, measles virus fusion (MVF) protein peptide (including, but not limited to SEQ ID NO: 11) or Tetanus Toxoid (TT3) peptide (including, but not limited to SEQ ID NO: 12)), and a linker (such as, for example, SEQ ID NO: 13) joining the SARS-CoV-2 B cell epitope to the Th epitope, wherein the one or more SARS-CoV-2 B cell epitopes consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. For example, disclosed herein are chimeric peptides for stimulating an immune response to a coronavirus spike protein wherein the peptide comprises the amino acid sequence as set forth in SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and/or SEQ ID NO: 23.

Also disclosed herein are coronavirus spike protein synthetic peptide for stimulating an immune response to a coronavirus spike protein comprising one or more of the sequences as set forth in SEQ ID NO: 24, SEQ ID NO: 25, SEQ ID NO: 26, SEQ ID NO: 27, SEQ ID NO: 28, SEQ ID NO: 29, or SEQ ID NO: 30.

In one aspect, disclosed herein are chimeric peptides comprising one or more coronavirus spike protein synthetic peptides of any preceding aspect (such as, for example, SEQ ID NO: 24, SEQ ID NO: 25, SEQ ID NO: 26, SEQ ID NO: 27, SEQ ID NO: 28, SEQ ID NO: 29, or SEQ ID NO: 30), further comprising a Th epitope (such as, for example, measles virus fusion (MVF) protein peptide (including, but not limited to SEQ ID NO: 11) or a tetanus toxoid (TT3) peptide (including, but not limited to SEQ ID NO: 12)), and a linker (such as, for example, SEQ ID NO: 13) joining the synthetic coronavirus spike protein peptide to the Th epitope. For example, disclosed herein are chimeric peptides comprising one or more coronavirus spike protein synthetic peptides, wherein the coronavirus spike protein synthetic peptide comprises the amino acid sequence as set forth in SEQ ID NO: 31, SEQ ID NO: 32, SEQ ID NO: 33, SEQ ID NO: 34, SEQ ID NO: 35, SEQ ID NO: 36, or SEQ ID NO: 37.

Also disclosed herein are modified coronavirus spike protein peptides for stimulating an immune response to a coronavirus spike protein comprising one or more of the sequences as set forth in SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47.

In one aspect, disclosed herein are chimeric peptides comprising one or more modified coronavirus spike protein peptides of any preceding aspect (such as, for example, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47), further comprising a Th epitope (such as, for example, measles virus fusion (MVF) protein peptide (including, but not limited to SEQ ID NO: 11) or a tetanus toxoid (TT3) peptide (including, but not limited to SEQ ID NO: 12)), and a linker (such as, for example, SEQ ID NO: 13) joining the modified coronavirus spike protein peptide to the Th epitope. For example, disclosed herein are chimeric peptides comprising a modified coronavirus spike protein peptide, wherein the modified coronavirus spike protein peptide comprises the amino acid sequence as set forth in SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57.

Also disclosed herein are chimeric peptides, synthetic peptides, and/or modified peptides of any preceding aspect wherein the amino acids comprising the SARS-CoV-2 B cell epitope are the D enantiomer and/or wherein the SARS-CoV-2 B cell epitope is acetylated.

In one aspect, disclosed herein are pharmaceutical compositions comprising one or more chimeric peptides or synthetic peptides of any preceding aspect and a pharmaceutically acceptable vehicle (such as, for example, an adjuvant including, but not limited to aluminium hydroxide, aluminium phosphate, potassium aluminium sulfate, calcium phosphate hydroxide, Freund's complete adjuvant, a water in oil adjuvant including, but not limited to, MONTANIDE®, Freund's incomplete adjuvant, iscoms, iscom matrix, ISCOMATRIX™ adjuvant, Matrix M™ adjuvant, Matrix C™ adjuvant, Matrix Q™ adjuvant, AbISCO®-100 adjuvant, AbISCO®-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative).

In one aspect, the pharmaceutical composition can further comprise a chimeric PD-1 or PD-L1 peptide comprising a PD-1 or PD-L1 B cell epitope (such as, for example, SEQ ID NO: 9 or SEQ ID NO: 10), a Th epitope (such as, for example, measles virus fusion (MVF) protein peptide (including, but not limited to SEQ ID NO: 11) or a tetanus toxoid (TT3) peptide (including, but not limited to SEQ ID NO: 12)), and a linker (such as, for example, SEQ ID NO: 13) joining the PD-1 and/or PD-L1 B cell epitope to the Th epitope (such as, for example (SEQ ID NO: 22 and/or SEQ ID NO: 23)

Also disclosed herein are antibodies that specifically binds to any of the chimeric, modified, or synthetic peptides of any preceding aspect.

In one aspect, disclosed herein are methods of treating, inhibiting, reducing, decreasing, ameliorating, and/or preventing a coronavirus infection (such as, for example, an infection with an avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, or middle east respiratory syndrome (MERS) coronavirus (CoV) (MERS-CoV), including, but not limited to the B1.351 variant, B.1.1.7 variant, and P.1 variant) in a subject comprising administering to the subject any of the peptides, antibodies, or compositions of any preceding aspect or any combination thereof.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows a drawing of the position of B-cell epitopes PK1, PK2, PK3, PK4, PK5, PK7, and PK8 relative to the SARS-CoV2 spike protein.

FIG. 1B shows the amino acid sequence of SARS-CoV2 B cell epitopes and chimeric peptides with tetanus toxoid (TT3) or measles virus fusion protein (MVF) Th epitope. Specifically shown are PK-1 (465-485) (SEQ ID NO: 2), TT3-PK1 (465-485)(SEQ ID NO: 14), MVF-PK1 (465-485)(SEQ ID NO: 15), PK-2 (402-419)(SEQ ID NO: 3), TT3-PK2 (402-419)(SEQ ID NO: 16), PK-3 (564-584)(SEQ ID NO: 4), TT3-PK3 (564-584)(SEQ ID NO: 17), PK-4 (773-790)(SEQ ID NO: 5), TT3-PK4 (773-790)(SEQ ID NO: 18), PK-5 (1146-1163)(SEQ ID NO: 6), TT3-PK5 (485-502)(SEQ ID NO: 19), PK-7 (485-502)(SEQ ID NO: 7), TT3-PK7 (773-790)(SEQ ID NO: 20), PK-8 (369-392)(SEQ ID NO: 8), TT3-PK8 (369-392)(SEQ ID NO: 21), PD-1 (92-110)(SEQ ID NO: 9), and TT3-PD-1 (92-110)(SEQ ID NO: 22).

FIG. 1C shows the immunization schemes in rabbits. Scheme of immunization with B-cell epitopes of SARS-CoV2 vaccines on New Zealand White rabbits. Rabbits were immunized with 1 mg of each TT3-peptide immunogens dissolved in dd H2O emulsified (1:1) in Montanide ISA 720 vehicle. The rabbits were boosted with the same doses with 3 weeks apart. Blood was collected via the central auricular artery in rabbits. And the terminal sera were collected at 3Y+3 which is 3 weeks after the last immunization.

FIG. 1D shows the immunogenicity of rabbits. Immunogenicity of rabbits immunized with B-cell epitopes were evaluated by ELISA. 200 ng/well peptide were used to coat the ELISA plates. Titers are defined as the highest dilution of sera with an absorbance value of 0.2 after subtracting the blank. The log 2 titers showed here means the highest dilutions were calculated by log 2 (eg. the highest dilution is 32000, log 2 value is 15 etc.) Only one rabbit immunized with PK7 showed relatively lower titer.

FIG. 2 shows the antigenicity profiles of each of the B cell epitopes PK1 (SEQ ID NO: 2), PK2 (SEQ ID NO: 3), PK3 (SEQ ID NO: 4), PK4 (SEQ ID NO: 5), PK5 (SEQ ID NO: 6), PK6 (SEQ ID NO: 2), PK7 (SEQ ID NO: 7), and PK8 (SEQ ID NO. 8). FIG. 2 also shows a cartoon of the SARS spike protein, the primary structure and amino acid sequence of the SARS spike protein (SEQ ID NO: 68), and the protein folding and epitopes.

FIGS. 3A and 3B show the immunization schemes in mice. Scheme of C57BL/6J (3A) and BALB/c (3B) wild type mice vaccination. The black wild type mice (10 mice/gp) 6-8 weeks old were immunized with designed peptide vaccine emulsified with ISA 720 vehicle. Mice were immunized three times and two or three weeks apart, 3 weeks after the third immunization (3Y+3) all the mice were terminated for the final bleed.

FIGS. 3C and 3D shows the immunogenicity of mice. Immunogenicity of mice immunized with B-cell epitopes were evaluated by ELISA at 2 weeks (3C) and 3 weeks (3D). 200 ng/well peptide were used to coat the ELISA plates. Titers are defined as the highest dilution of sera with an absorbance value of 0.2 after subtracting the blank. The log 2 titers showed here means the highest dilutions were calculated by log 2 (eg. the highest dilution is 32000, log 2 value is 15 etc.).

FIG. 4 shows antibody isotyping from mice sera for chimeric peptides 2 weeks and 3 weeks post immunization.

FIGS. 5A and 5B show recombinant protein activity against spike, S1 (5A) and S2 (5B).

FIG. 5C shows the overlap of SARS-CoV2 (residues 381-520 of SEQ ID NO: 1) and SARS (residues 368-506 of SEQ ID NO: 68) B cell epitopes and peptide vaccine locations.

FIG. 5D shows the cross-reactivity of antibodies to PK1, PK2 and PK6 from both SARS-CoV2 and SARS.

FIG. 6 shows results from a representative SARS-CoV2 pseudovirus neutralization assay.

FIGS. 7A and 7B show the generation of K18-hACE2 transgenic mice (7A) and immunization schedule (7B).

FIG. 7C shows the immunogenicity titers in transgenic mice against each peptide epitopes were evaluated by ELISA. 200 ng/well peptide were used to coat the 96-well ELISA plates. Titers are defined as the highest dilution of sera with an absorbance value of 0.2 after subtracting the blank.

FIG. 7D shows the percentage of mice body weight change after inoculated with SARS-CoV2 virus.

FIG. 7E shows the results of SARSS-CoV2 challenge experiments in K18hACE2 transgenic mice.

FIGS. 8A and 8B show that the peptide vaccine protects mice body weight loss and viral RNA load. Only mice immunized with PK5 showed significant lower body weight loss (8A) and lower viral RNA loading (8B) versus PBS control. All TG mice were inoculated with 240,000 pfu/mouse SARS-CoV2 (USA-WA1-2020 strain).

FIGS. 9A, 9B, and 9C show the effects of SARS-CoV2 infection. FIG. 11A shows immunogenicity determined by ELISA. Antibodies were boosted in both male and female immunized transgenic mice. (11B and 11C) Mice body weight were monitored daily after virus challenge. 50% of mice in each group were sacrificed on day 2 post inoculation, all left over mice were sacrificed on day 4 post inoculation. Day0 body weight of each mouse was used as base line.

FIGS. 10A, 10B, 10C, 10D, 10E, and 10F show the ability of anti-PK-1 (10A and 10B), anti-PK-2 (10C and 10D), and anti-PK-7 (10E and 10F) antibodies to bind PK-1, PK-2, and PK-7 mutants, respectively. FIG. 10B shows the amino acid sequence of PK1 (465-485)(SEQ ID NO: 2), TT3-PK-1(465-485)(SEQ ID NO: 14), MVF-PK1 (465-485)(SEQ ID NO. 15), PK-6 (also known as MVF-PK-1)(SEQ ID NO: 15) and mutant peptides PK1.1 (SEQ ID NO: 48), PK1.2 (SEQ ID NO: 49), PK-1.3 (SEQ ID NO: 50), and PK-1.4 (SEQ ID NO: 51). FIG. 10D shows the amino acid sequence of PK-2 (402-419)(SEQ ID NO: 3), TT3-PK2 (402-419)(SEQ ID NO: 16) and the mutant peptide PK-2.1 (SEQ ID NO: 42). FIG. 10F shows the amino acid sequence of PK-7 (485-502)(SEQ ID NO: 7) and TT3-PK7 (773-790)(SEQ ID NO: 20) as well as mutant peptides PK-7.1 (485-502)(SEQ ID NO: 43), PK-7.2 (485-502)(SEQ ID NO: 44), PK-7.3 (485-502)(SEQ ID NO: 45), PK-7.4 (485-502)(SEQ ID NO: 46), and PK-7.5 (485-502)(SEQ ID NO: 47).

FIGS. 11A and 11B show the percent body weight change following virus challenge. FIG. 11A shows a comparison in male mice between control and PK5 immunized mice. Two-way ANOVA analysis p<0.01. Bonferroni's multiple comparisons p<0.05 both at day 2 and day 4. FIG. 11B shows a comparison in male mice between control, PK1, and PK5 immunized mice. Two-way ANOVA analysis p<0.01. Bonferroni's multiple comparisons p<0.05 both at day 2 and day 3.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. Embodiments defined by each of these transition terms are within the scope of this invention.

An “effective amount” is an amount sufficient to effect beneficial or desired results. An effective amount can be administered in one or more administrations, applications or dosages.

The terms “treat”, “treating”, “treatment” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. In some instances, the terms “treat”, “treating”, “treatment” and grammatical variations thereof, include partially or completely reducing the size of a tumor, reducing the number of tumors, and reducing the severity/metastatic ability of a tumor as compared with prior to treatment of the subject or as compared with the incidence of such symptom in a general or study population.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound. As used herein, a “wt. %” or “weight percent” or “percent by weight” of a component, unless specifically stated to the contrary, refers to the ratio of the weight of the component to the total weight of the composition in which the component is included, expressed as a percentage.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular synthetic or chimeric PD-L1 peptide is disclosed and discussed and a number of modifications that can be made to a number of molecules including the synthetic or chimeric PD-L1 peptide are discussed, specifically contemplated is each and every combination and permutation of the synthetic or chimeric PD-L1 peptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are chimeric peptides for stimulating an immune response to a coronavirus spike protein comprising one or more SARS-CoV-2 B cell epitopes, a T helper (Th) epitope, and a linker joining the SARS-CoV-2 B cell epitope to the Th epitope, wherein the one or more SARS-CoV-2 B cell epitopes consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

The Th epitope can be from about 14 to about 22, more preferably about 15 to 21, most preferably 16 amino acids in length. Preferably, the Th cell epitope has one of the following amino acid sequences provided in Table 1.

TABLE 1

| Peptide | | |
|---|---|---|
| Designation | Sequence | SEQ ID NO: |
| MVF | KLLSLIKGVIVHRLEGVE | 11 |
| TT | NSVDDALINSTIYSYFPSV | 62 |
| TT1 | PGINGKAIHLVNNQSSE | 63 |
| P2 | QYIKANSKFIGITEL | 64 |
| P30 (TT3) | FNNFTVSFWLRVPKVSASHLE | 12 |
| MVF (natural) | LSEIKGVIVHRLEGV | 65 |
| HBV | FFLLTRILTIPQSLN | 66 |
| CSP | TCGVGVRVRSRVNAANKKPE | 67 |

To join the coronavirus spike protein peptide (including synthetic and modified peptides) and the Th cell epitope, an amino acid linker can be used. Preferably the linker is a peptide of from about 2 to about 15 amino acids, more preferably from about 2 to about 10 amino acids, most preferably from about 2 to about 6 amino acids in length. The most preferred linker comprises the amino acid sequence Gly-Pro-Ser-Leu (SEQ ID NO: 13). Thus, in one aspect, also disclosed herein are chimeric peptides comprising any of the coronavirus spike protein B cell epitopes, modified coronavirus spike protein peptides, and/or corona virus spike protein synthetic peptides disclosed herein, further comprising a Th epitope (such as, for example, measles virus fusion (MVF) protein peptide (including, but not limited to SEQ ID NO: 11) or a tetanus toxoid(TT3) peptide (including, but not limited to SEQ ID NO: 12)), and a linker (such as, for example, SEQ ID NO: 13) joining the coronavirus spike protein B cell epitope, modified coronavirus spike protein peptide, and/or corona virus spike protein synthetic peptide to the Th epitope. For example, disclosed herein, in one aspect, are chimeric peptides for stimulating an immune response to a coronavirus spike protein comprising one or more SARS-CoV-2 B cell epitopes, a T helper (Th) epitope (such as, for example, measles virus fusion (MVF) protein peptide (including, but not limited to SEQ ID NO: 11) or a tetanus toxoid (TT3) peptide (including, but not limited to SEQ ID NO: 12)), and a linker (such as, for example, SEQ ID NO: 13) joining the SARS-CoV-2 B cell epitope to the Th epitope, wherein the one or more SARS-CoV-2 B cell epitopes consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. For example, disclosed herein are chimeric peptides for stimulating an immune response to a coronavirus spike protein wherein the peptide comprises the amino acid sequence as set forth in SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and/or SEQ ID NO: 23.

In one aspect, it is also understood and herein contemplated that the disclosed B cell epitopes can be engineered to comprise one or more substitutions (i.e., modified coronavirus spike protein peptides) or be in reverse order (retro, referred to herein as coronavirus spike protein synthetic peptides). For example, the modified coronavirus spike protein peptide can comprise the sequence SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47. Also disclosed herein are synthetic peptide for stimulating an immune response to a coronavirus spike protein comprising one or more of the sequences as set forth in SEQ ID NO: 24, SEQ ID NO: 25, SEQ ID NO: 26, SEQ ID NO: 27, SEQ ID NO: 28, SEQ ID NO: 29, or SEQ ID NO: 30.

As with the SARS-CoV2 B cell epitopes, the synthetic and modified peptides may be used as a B cell epitope for a chimeric coronavirus spike protein peptide, said chimeric peptide further comprising a Th epitope (such as, for example, measles virus fusion (MVF) protein peptide (including, but not limited to SEQ ID NO: 11) or a tetanus toxoid(TT3) peptide (including, but not limited to SEQ ID NO: 12)), and a linker (such as, for example, SEQ ID NO: 13) joining the coronavirus spike protein B cell epitope, modified coronavirus spike protein peptide, and/or corona virus spike protein synthetic peptide to the Th epitope. Thus, in one aspect, disclosed herein are chimeric peptides comprising one or more coronavirus spike protein synthetic peptides (such as, for example, SEQ ID NO: 24, SEQ ID NO: 25, SEQ ID NO: 26, SEQ ID NO: 27, SEQ ID NO: 28, SEQ ID NO: 29, or SEQ ID NO: 30), further comprising a Th epitope (such as, for example, measles virus fusion (MVF) protein peptide (including, but not limited to SEQ ID NO: 11) or Tetanus Toxoid (TT3) peptide (including, but not limited to SEQ ID NO: 12)), and a linker (such as, for example, SEQ ID NO: 13) joining the synthetic coronavirus spike protein peptide to the Th epitope. For example, disclosed herein are chimeric peptides comprising one or more coronavirus spike protein synthetic peptides, wherein the coronavirus spike protein synthetic peptide comprises the amino acid sequence as set forth in SEQ ID NO: 31, SEQ ID NO: 32, SEQ ID NO: 33, SEQ ID NO: 34, SEQ ID NO: 35, SEQ ID NO: 36, or SEQ ID NO: 37.

Also disclosed herein are one or more modified coronavirus spike protein peptides (such as, for example, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47), further comprising a Th epitope (such as, for example, measles virus fusion (MVF) protein peptide (including, but not limited to SEQ ID NO: 11) or Tetanus Toxoid (TT3) peptide (including, but not limited to SEQ ID NO: 12)), and a linker (such as, for example, SEQ ID NO: 13) joining the modified coronavirus spike protein peptide to the Th epitope. For example, disclosed herein are chimeric peptides comprising a modified coronavirus spike protein peptide, wherein the modified coronavirus spike protein peptide comprises the amino acid sequence as set forth in SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57.

It is understood and herein contemplated that any of the chimeric peptides, modified coronavirus spike protein peptides, and/or coronavirus spike protein synthetic peptides can be formulated as a vaccine or pharmaceutical composition which can be administered therapeutically or prophylactically to a subject having or at risk of a coronavirus infection.

In one aspect, the vaccine or pharmaceutical composition can comprise one or more chimeric peptides, modified coronavirus spike protein peptides, and/or coronavirus spike protein synthetic peptides disclosed herein and a pharmaceutically acceptable vehicle (such as, for example, a vehicle that is biodegradable including, but not limited to an emulsion comprising a pharmaceutically acceptable adjuvant. As used herein, the term “adjuvant” typically refers to a class of substance that can increase the magnitude of the immune response elicited by any of the chimeric coronavirus peptides, modified coronavirus spike protein peptides, and/or coronavirus spike protein synthetic peptides beyond that which would be expected, either from the chimeric peptides alone or from the chimeric peptides as herein described in the absence of an adjuvant.

Suitable adjuvants will be known to persons skilled in the art. Non-limiting examples of suitable adjuvants include aluminium salts (e.g. aluminium hydroxide, aluminium phosphate and potassium aluminium sulfate (also referred to as Alum)), liposomes, virosomes, water-in-oil or oil-in-water emulsions (e.g. Freund's adjuvant, Montanide®, MF59® and AS03), 3-O-desacyl-4′-monophosphoryl lipid A (MPL) and adjuvants containing MPL (e.g. AS01, AS02 and AS04) and saponin-based adjuvants. Saponin-based adjuvants include saponins or saponin derivatives from, for example, Quillaja saponaria, Panax ginseng Panax nologinseng, Panax quinquefolium, Platycodon grandiflorum, Polygala senega, Polygala tenuifolia, Quillaja brasiliensis, Astragalus membranaceus and Achyranthes bidentata. Exemplary saponin-based adjuvants include iscoms, iscom matrix, ISCOMATRIX™ adjuvant, Matrix M™ adjuvant, Matrix C™ adjuvant, Matrix Q™ adjuvant, AbISCO®-100 adjuvant, AbISCO®-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative. The vaccine composition as herein described can also be associated with immumodulatory agents, including, for example, cytokines, chemokines and growth factors. Mixtures of two or more adjuvants within the same vaccine composition are also contemplated herein. In an embodiment, the adjuvant is water in oil adjuvant Montanide (ISA720). For example, disclosed herein are pharmaceutical compositions comprising one or more chimeric peptides, modified coronavirus spike protein peptides, and/or coronavirus spike protein synthetic peptides (for example any of the peptides as set forth in SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57) further comprising an adjuvant selected from the group consisting of aluminium hydroxide, aluminium phosphate, potassium aluminium sulfate, calcium phosphate hydroxide, Freund's complete adjuvant, Montanide®, Freund's incomplete adjuvant, iscoms, iscom matrix, ISCOMATRIX™ adjuvant, Matrix M™ adjuvant, Matrix C™ adjuvant, Matrix Q™ adjuvant, AbISCO®-100 adjuvant, AbISCO®-300 adjuvant, ISCOPREP™, an ISCOPREP™ derivative, adjuvant containing ISCOPREP™ or an ISCOPREP™ derivative, QS-21, a QS-21 derivative, and an adjuvant containing QS-21 or a QS21 derivative.

In one aspect, disclosed herein are antibodies that specifically bind to any of the chimeric peptides, modified coronavirus spike protein peptides, and/or coronavirus spike protein synthetic peptides disclosed herein.

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Peptides

### a) Protein and Peptide Variants

As discussed herein there are numerous variants of the chimeric coronavirus spike protein peptides, modified coronavirus spike protein peptides, and coronavirus spike protein synthetic peptides that are known and herein contemplated. In addition, to the known functional coronavirus spike protein strain variants there are derivatives of the chimeric coronavirus spike protein peptides, modified coronavirus spike protein peptides, and coronavirus spike protein synthetic peptides which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 3
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn;Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl: (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues.

Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% identity to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that peptide or protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 2 and Table 3. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. In other words, contemplated herein is the inverso (i.e., the D-amino acid substitution) of any disclosed sequence. Cysteine residues can be used to cyclize or attach two or more peptides together. This can be beneficial to constrain peptides into particular conformations. In one aspect, disclosed herein are synthetic coronavirus spike protein peptides comprising one or more of the sequences as set forth in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47 wherein the amino acids of the peptide are the D enantiomer.

In one aspect, the disclosed synthetic peptides can be in reverse order such that the amino to carboxy end of the peptide is reversed (i.e., the retro sequence). In one aspect, disclosed herein are the retro sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, which comprises, SEQ ID NO: 24, SEQ ID NO: 25, SEQ ID NO: 26, SEQ ID NO: 27, SEQ ID NO: 28, SEQ ID NO: 29, or SEQ ID NO: 30, respectively. These retro sequences can also have the mirror conformation of the base sequence. In one aspect, the retro sequence can also comprise a D amino acid substitution (i.e., the retro-inverso) sequence. Thus, disclosed herein are coronavirus spike protein synthetic peptides comprising one or more of the sequences as set forth in SEQ ID NO: 24, SEQ ID NO: 25, SEQ ID NO: 26, SEQ ID NO: 27, SEQ ID NO: 28, SEQ ID NO: 29, or SEQ ID NO: 30; wherein the amino acids of the peptide are the D enantiomer.

It is understood that any of the D amino acid substituted synthetic peptides disclosed herein can be used in as the coronavirus spike protein B cell epitope in the disclosed coronavirus spike protein chimeric peptides. For example, disclosed herein are chimeric coronavirus spike protein peptides comprising one or more coronavirus spike protein B cell epitopes, a T helper (Th) epitope, and a linker joining the coronavirus spike protein B cell epitope to the Th epitope, wherein the one or more coronavirus spike protein B cell epitopes consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10; and wherein the amino acids of the peptide are the D enantiomer. In one aspect, disclosed herein are chimeric coronavirus spike protein synthetic peptides, wherein the peptide comprises the amino acid sequence as set forth in SEQ ID NO: 24, SEQ ID NO: 25, SEQ ID NO: 26, SEQ ID NO: 27, SEQ ID NO: 28, SEQ ID NO: 29, SEQ ID NO: 30; and wherein the amino acids of the coronavirus spike protein synthetic peptide are the D enantiomer. Also disclosed are chimeric modified coronavirus spike protein peptides, wherein the peptide comprises the amino acid sequence as set forth in SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47; and wherein the amino acids of the peptide are the D enantiomer.

### 3. Pharmaceutical Carriers/Delivery of Pharmaceutical Products

As described above, the chimeric peptides, modified coronavirus spike protein peptides, and/or coronavirus spike protein synthetic peptides disclosed herein can also be administered in vivo in a pharmaceutically acceptable carrier. Thus, in one aspect, disclosed herein are pharmaceutical composition comprising any one or more of the chimeric peptides, modified coronavirus spike protein peptides, and/or coronavirus spike protein synthetic peptides peptides as set forth herein.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

It is understood and herein contemplated that the disclosed chimeric peptides, modified coronavirus spike protein peptides, and/or coronavirus spike protein synthetic peptides comprising pharmaceutical compositions are particularly useful in the treatment, inhibition, reduction, decrease, amelioration, and/or prevention of a coronavirus infection.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993): Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The chimeric peptides, modified coronavirus spike protein peptides, coronavirus spike protein synthetic peptides, chimeras, and antibodies disclosed herein that inhibit the interaction of a coronavirus spike protein and ACE2 can be administered prophylactically to patients or subjects who are at risk for developing a coronaviral infection.

Other molecules or antibodies that interact with PD-1 or PD-L1 to inhibit PD-1/PD-L1 interactions (for example, Pembrolixumab and nivolumab) can be used in combination with the disclosed synthetic PD-L1 peptides, chimeric PD-L1 peptides, or anti-PD-L1 antibodies to treat, inhibit, reduce, decrease, ameliorate, and/or prevent a coronavirus infection.

### 4. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a coronavirus spike protein is inhibited from interacting with angiotensin converting enzyme 2 (ACE2). Antibodies that bind SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57 involved in the interaction between a coronavirus spike protein and ACE2 are also disclosed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies which maintain coronavirus spike protein binding activity or bind SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57 are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

### (4) Administration of Antibodies

Administration of the antibodies can be done as disclosed herein. Nucleic acid approaches for antibody delivery also exist. The broadly neutralizing anti-coronavirus spike protein antibodies and antibody fragments (including any antibody that binds to SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO. 57) can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### C. Method of Treating Disease

It is understood that the disclosed chimeric peptides, modified coronavirus spike protein peptides, coronavirus spike protein synthetic peptides, antibodies binding one or more of said peptides, antibodies binding one or more coronavirus spike protein B cell epitopes, and pharmaceutical compositions comprising any of the peptides or antibodies can be administered for the treatment, inhibition, reduction, decrease, amelioration, and/or prevention of coronavirus infections. Due to the conserved nature of the coronavirus spike protein (see sequence alignment included herein in Table 4 below), it is understood and herein contemplated that the coronavirus being treated can be any coronavirus including, but not limited to avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, or middle east respiratory syndrome (MERS) coronavirus (CoV) (MERS-CoV), including, but not limited to the B1.351 variant, B.1.1.7 variant, and P.1 variant.

TABLE 4
CLUSTAL O(1.2.4) multiple sequence alignment

| | | |
|---|---|---|
| TR\|A0A6H1PJZ3\|A0A6H1PJZ3_SARS2 | ----MFVFLVLLPLV-S----------SQCVN--LTT--RTQLPPAY--TNSFTRGVYYP | 39 |
| SP\|P59594\|SPIKE_SARS | ----MFIFLLFLTLT-S----------GSDLD--RCTTFDDVQAPNYTQHTSSMRGVYYP | 43 |
| SP\|K9N5Q8\|SPIKE_MERS1 | MIHSVFLLMFLLTPTESYVDVGPDSVKSACIEVDIQQTFFDKTWPRP-IDVSKADGIIYP | 59 |
| | :*:::.:* . * . :: * * *: ** | |
| TR\|A0A6H1PJZ3\|A0A6H1PJZ3_SARS2 | DKVFRSSVLHSTQDLFLPFFSNVT---WFHAIHVSGTNGTKR----FDNPVLPENDGVYF | 92 |
| SP\|P59594\|SPIKE_SARS | DEIFRSDTLYLTQDLFLPFYSNVT---GFHTIN-------HT----FGNPVIPFKDGIYF | 89 |
| SP\|K9N5Q8\|SPIKE_MERS1 | QGRTYSNITITYQGLF-PYQGDHGDMYVYSAGHATGTTPQKLFVANYSQDVKQFANGFVV | 118 |
| | : *. *.** *: .: : : : : :.: * * :*. . | |
| TR\|A0A6H1PJZ3\|A0A6H1PJZ3_SARS2 | ASTE-----------------KSNIIRGWIFGTTLDSKTQ---------SLLIVNNATNV | 126 |
| SP\|P59594\|SPIKE_SARS | AATE-----------------KSNVVRGWVFGSTMNNKSQ---------SVIIINNSTNV | 123 |
| SP\|K9N5Q8\|SPIKE_MERS1 | RIGAAANSTGTVIISPSTSATIRKIYPAFMLGSSVGNFSDGKMGRFFNHTLVLLPDGCGT | 178 |
| | :: .:::*:::.. :: ::::: :. .. | |
| TR\|A0A6H1PJZ3\|A0A6H1PJZ3_SARS2 | VIKVCEFQFCNDPFLGVYYHKNN---------------------KSWMESEFRVYSSANN | 165 |
| SP\|P59594\|SPIKE_SARS | VIRACNFELCDNPFFAVSKPMGT-------------------------QTHTMIFDNAFN | 158 |
| SP\|K9N5Q8\|SPIKE_MERS1 | LLRAF--YCILEPRSGNHCPAGNSYTSFATYHTPATDCSDGNYNRNASLNSFKEYFNLRN | 236 |
| | :::. :* . .. . : . * | |
| TR\|A0A6H1PJZ3\|A0A6H1PJZ3_SARS2 | CTFEYVSQPFLMDLEGKQGNFKNLREFVFKNIDGYFKIYSKHTPINLVRDLPQGFSALEP | 225 |
| SP\|P59594\|SPIKE_SARS | CTFEYISDAFSLDVSEKSGNFKHLREFVFKNKDGFLYVYKGYQPIDVVRDLPSGFNTLKP | 218 |
| SP\|K9N5Q8\|SPIKE_MERS1 | CTFMYTYNITEDEILEWFGITQTAQG-VHLFSSRYVDLYGGN---------------MFQ | 280 |
| | *** * : :: * : : *. . :. :* : | |
| TR\|A0A6H1PJZ3\|A0A6H1PJZ3_SARS2 | LVDLPIGINITRFQTLLALHRSYLTPGDSSSGWTAGAAAYYVGYLQPRTFLLKYNENGTI | 285 |
| SP\|P59594\|SPIKE_SARS | IFKLPLGINITNFRAILTAFS------PAQDIWGTSAAAYFVGYLKPTTFMLKYDENGTI | 272 |
| SP\|K9N5Q8\|SPIKE_MERS1 | FATLPVYDTIKYYSIIPHSIR---SIQSDRKAW----AAFYVYKLQPLTFLLDFSVDGYI | 333 |
| | : **: .*. : : . * **::* *:* **:*.:. :* * | |
| TR\|A0A6H1PJZ3\|A0A6H1PJZ3_SARS2 | TDAVDCALDPLSETKCTLKSFTVEKGIYQTSNFRVQPTESIVRFPNITNLCPFGEVFNAT | 345 |
| SP\|P59594\|SPIKE_SARS | TDAVDCSQNPLAELKCSVKSFEIDKGIYQTSNFRVVPSGDVVRFPNITNLCPFGEVFNAT | 332 |
| SP\|K9N5Q8\|SPIKE_MERS1 | RRAIDCGFNDLSQLHCSYESFDVESGVYSVSSFEAKPSGSVVEQAEG-VECDFSPLLSG- | 391 |
| | *:**. : *:: :*: :** ::.*:*..*.*.. *: .:*. : * *. ::.. | |
| TR\|A0A6H1PJZ3\|A0A6H1PJZ3_SARS2 | RFASVYAWNRKRISNCVADYSVLYNSASFSTFKCYGVSPTKLNDLCFTNVYADSFVIRGD | 405 |
| SP\|P59594\|SPIKE_SARS | KFPSVYAWERKKISNCVADYSVLYNSTFFSTFKCYGVSATKLNDLCFSNVYADSFVVKGD | 392 |
| SP\|K9N5Q8\|SPIKE_MERS1 | TPPQVYNFKRLVFTNCNYNLTKLLSLFSVNDFTCSQISPAAIASNCYSSLILDYFSYPLS | 451 |
| | .** ::* ::** : : * . .. *.* :* : : . *::.: * * . | |
| TR\|A0A6H1PJZ3\|A0A6H1PJZ3_SARS2 | EVRQIAPGQTGKIADYNYKLPDDFTGCVIAWNSNNLDSKVGGNYNYLYRLFRKSNLKPFE | 465 |
| SP\|P59594\|SPIKE_SARS | DVRQIAPGQTGVIADYNYKLPDDFMGCVLAWNTRNIDATSTGNYNYKYRYLRHGKLRPFE | 452 |
| SP\|K9N5Q8\|SPIKE_MERS1 | MKSDLSVSSAGPISQFNYKQSFSNPTCLILATVPHNLTTITKPLKYSYINKCSRFLSDDR | 511 |
| | ::: ..:* *:::*** . *:: . : :. :* * * . | |
| TR\|A0A6H1PJZ3\|A0A6H1PJZ3_SARS2 | RDISTEIYQAGSTPCNGVEGFNCYF-----------PLQSYGFQPTNGVGYQPYRVVVLS | 514 |
| SP\|P59594\|SPIKE_SARS | RDISNVPFSPDGKPCTP-PALNCYW-----------PLNDYGFYTTTGIGYQPYRVVVLS | 500 |
| SP\|K9N5Q8\|SPIKE_MERS1 | TEVPQLVNANQYSPCVSIVPST-VWEDGDYYRKQLSPLEGGGWLVASGSTVAMTEQLQMG | 570 |
| | :: .** . : **:. *: :.* . : :. | |
| TR\|A0A6H1PJZ3\|A0A6H1PJZ3_SARS2 | FELL----HAPATVCGP-----KKSTNLVKNKCVNFNFNGLIGTGVLTESNKKFLPFQQF | 565 |
| SP\|P59594\|SPIKE_SARS | FELL----NAPATVCGP-----KLSTDLIKNQCVNFNFNGLTGTGVLTPSSKRFQPFQQF | 551 |
| SP\|K9N5Q8\|SPIKE_MERS1 | FGITVQYGTDTNSVCPKLEFANDTKIASQLGNCVEYSLYGVSGRGVFQNCTAVGVRQQRF | 630 |
| | * : :** . . .:**::.: *::* **: .. *:* | |
| TR\|A0A6H1PJZ3\|A0A6H1PJZ3_SARS2 | GRDIADTT-DAVRDPQTLEILDITPCSFGGVSVITPGTNTSNQVAVLYQGVNCTEVPVAI | 624 |
| SP\|P59594\|SPIKE_SARS | GRDVSDFT-DSVRDPKTSEILDISPCSFGGVSVITPGTNASSEVAVLYQDVNCTDVSTAI | 610 |
| SP\|K9N5Q8\|SPIKE_MERS1 | VYDAYQNLVGYYSD--DGNYYCLRACVSVPVSVIYD--KETKTHATLFGSVACEHISSTM | 686 |
| | * : . * : : * **** : :. *.*: .* * .: :: | |
| TR\|A0A6H1PJZ3\|A0A6H1PJZ3_SARS2 | HADQLT--PTWRVYSTGSNVFQTRAGCLIGAEHVN-NSYECDIPIGAGICASYQTQT-NS | 680 |
| SP\|P59594\|SPIKE_SARS | HADQLT--PAWRIYSTGNNVFQTQAGCLIGAEHVD-TSYECDIPIGAGICASYHTVS-L- | 665 |
| SP\|K9N5Q8\|SPIKE_MERS1 | SQYSRSTRSMLKRRDSTYGPLQTPVGCVLGLVNSSLFVEDCKLPLGQSLCALPDTPSTLT | 746 |
| | . : : .: . :** .**::* : . :*.:*:* .:** .* : | |
| TR\|A0A6H1PJZ3\|A0A6H1PJZ3_SARS2 | PRRARSVASQSI---IAYTMSLGAENSVAYSNNSIAIPTNFTISVTTEILPVSMTKTSVD | 737 |
| SP\|P59594\|SPIKE_SARS | ---LRSTSQKSI---VAYTMSLGADSSIAYSNNTIAIPTNFSISITTEVMPVSMAKTSVD | 719 |
| SP\|K9N5Q8\|SPIKE_MERS1 | PRSVRSVPGEMRLASIAFNHPIQV-DQLNSSYFKLSIPTNFSFGVTQEYIQTTIQKVTVD | 805 |
| | **. : :*:. : . ..: * .::*****::.:* * : .:: *.:** | |
| TR\|A0A6H1PJZ3\|A0A6H1PJZ3_SARS2 | CTMYICGDSTECSNLLLQYGSFCTQLNRALTGIAVEQDKNTQEVFAQVKQIYKTPPIKDF | 797 |
| SP\|P59594\|SPIKE_SARS | CNMYICGDSTECANLLLQYGSFCTQLNRALSGIAAEQDRNTREVFAQVKQMYKTPTLKYF | 779 |
| SP\|K9N5Q8\|SPIKE_MERS1 | CKQYVCNGFQKCEQLLREYGQFCSKINQALHGANLRQDDSVRNLFASVKSSQSSPIIPGF | 865 |
| | *. *:*.. :* :** :**.**:::*:** * .** ..:::**.**. .:* : * | |
| TR\|A0A6H1PJZ3\|A0A6H1PJZ3_SARS2 | GGF-NFSQILPDP---SKPSKRSFIEDLLFNKVTLADAGFIKQYGDCL--GDIAARDLIC | 851 |
| SP\|P59594\|SPIKE_SARS | GGF-NFSQILPDP---LKPTKRSFIEDLLFNKVTLADAGFMKQYGECL--GDINARDLIC | 833 |
| SP\|K9N5Q8\|SPIKE_MERS1 | GGDFNLTLLEPVSISTGSRSARSAIEDLLFDKVTIADPGYMQGYDDCMQQGPASARDLIC | 925 |
| | ** *:: : * . : ** ******:***:** *::: *.:*: * ****** | |
| TR\|A0A6H1PJZ3\|A0A6H1PJZ3_SARS2 | AQKFNGLTVLPPLLTDEMIAQYTSALLAGTITSGWTFGAGAALQIPFAMQMAYRFNGIGV | 911 |
| SP\|P59594\|SPIKE_SARS | AQKFNGLTVLPPLLTDDMIAAYTAALVSGTATAGWTFGAGAALQIPFAMQMAYRFNGIGV | 893 |
| SP\|K9N5Q8\|SPIKE_MERS1 | AQYVAGYKVLPPLMDVNMEAAYTSSLLGSIAGVGWTAGLSSFAAIPFAQSIFYRLNGVGI | 985 |
| | ** . * .*****: :* * **::*:.. *** * .: **** .: **:**:*: | |
| TR\|A0A6H1PJZ3\|A0A6H1PJZ3_SARS2 | TQNVLYENQKLIANQFNSAIGKIQDSLSSTASALGKLQDVVNQNAQALNTLVKQLSSNFG | 971 |
| SP\|P59594\|SPIKE_SARS | TQNVLYENQKQIANQFNKAISQIQESLTTTSTALGKLQDVVNQNAQALNTLVKQLSSNFG | 953 |
| SP\|K9N5Q8\|SPIKE_MERS1 | TQQVLSENQKLIANKFNQALGAMQTGFTTTNEAFHKVQDAVNNNAQALSKLASELSNTFG | 1045 |
| | **:** **** ***:**.*:. :* .:::* *: *:**.**:*****..*..:**..** | |
| TR\|A0A6H1PJZ3\|A0A6H1PJZ3_SARS2 | AISSVLNDILSRLDKVEAEVQIDRLITGRLQSLQTYVTQQLIRAAEIRASANLAATKMSE | 1031 |
| SP\|P59594\|SPIKE_SARS | AISSVLNDILSRLDKVEAEVQIDRLITGRLQSLQTYVTQQLIRAAEIRASANLAATKMSE | 1013 |
| SP\|K9N5Q8\|SPIKE_MERS1 | AISASIGDIIQRLDVLEQDAQIDRLINGRLTTLNAFVAQQLVRSESAALSAQLAKDKVNE | 1105 |
| | ***: :.**:.*** :* :.******.*** :*:::*:***:*: . **:** *:.* | |
| TR\|A0A6H1PJZ3\|A0A6H1PJZ3_SARS2 | CVLGQSKRVDFCGKGYHLMSFPQSAPHGVVFLHVTYVPAQEKNFTTAPAICHDGKA---H | 1088 |
| SP\|P59594\|SPIKE_SARS | CVLGQSKRVDFCGKGYHLMSFPQAAPHGVVFLHVTYVPSQERNFTTAPAICHEGKA---Y | 1070 |
| SP\|K9N5Q8\|SPIKE_MERS1 | CVKAQSKRSGFCGQGTHIVSFVVNAPNGLYFMHVGYYPSNHIEVVSAYGLCDAANPTNCI | 1165 |
| | ** .**** .***:* *::** **:*: *:** * *::. :..:* .:*. .: | |
| TR\|A0A6H1PJZ3\|A0A6H1PJZ3_SARS2 | FPREGVFVSN-----GTHWFVTQRNFYEPQIITTDNTFVSGNCDVVIGIVNNTVYDPLQ- | 1142 |
| SP\|P59594\|SPIKE_SARS | FPREGVFVFN-----GTSWFITQRNFFSPQIITTDNTFVSGNCDVVIGIINNTVYDPLQ- | 1124 |
| SP\|K9N5Q8\|SPIKE_MERS1 | APVNGYFIKTNNTRIVDEWSYTGSSFYAPEPITSLNTKYVAPQVTYQN-ISTNLPPPLLG | 1224 |
| | * :* *: . * * .*: *: **: ** . . . :...: ** | |
| TR\|A0A6H1PJZ3\|A0A6H1PJZ3_SARS2 | -PELDSFKEELDKYFKNHTSPDVDLGDISGINASVVNIQKEIDRINEVAKNLNESLIDLQ | 1201 |
| SP\|P59594\|SPIKE_SARS | -PELDSFKEELDKYFKNHTSPDVDLGDISGINASVVNIQKEIDRLNEVAKNLNESLIDLQ | 1183 |
| SP\|K9N5Q8\|SPIKE_MERS1 | NSTGIDFQDELDEFFKNVSTSIPNFGSLTQINTTLLDLTYEMLSLQQVVKALNESYIDLK | 1284 |
| | .*::***::*** :: ::*.:: **:::::: *: *::*.* **** ***: | |
| TR\|A0A6H1PJZ3\|A0A6H1PJZ3_SARS2 | ELGKYEQYIKWPWYIWLGFIAGLIAIVMVTIMLCCMTSCCSCLKGCCSCGSCCKF-DEDD | 1260 |
| SP\|P59594\|SPIKE_SARS | ELGKYEQYIKWPWYVWLGFIAGLIAIVMVTILLCCMTSCCSCLKGACSCGSCCKF-DEDD | 1242 |
| SP\|K9N5Q8\|SPIKE_MERS1 | ELGNYTYYNKWPWYIWLGFIAGLVALALCVFFILCCTGCGTNCMGKLKCNRCCDRYEEYD | 1344 |
| | ***:* * *****:********:*:.: .::: * *.* : * .*. **. :* * | |
| TR\|A0A6H1PJZ3\|A0A6H1PJZ3_SARS2 | SEPVLKGVKLHYT (SEQ ID NO: 1) | 1273 |
| SP\|P59594\|SPIKE_SARS | SEPVLKGVKLHYT (SEQ ID NO: 68) | 1255 |
| SP\|K9N5Q8\|SPIKE_MERS1 | LEPHKVHVH---- (SEQ ID NO: 69) | 1353 |
| | ** *: | |

SARS2 v. SARS1 Homology
Waterman-Eggert score: 6650; 1535.8 bits; E(1) < 0
76.0% identity (91.5% similar) in 1277 aa overlap (1-1273; 1-1255)
SARS2 v. MERS
Waterman-Eggert score: 1958; 482.9 bits; E(1) < 7.6e-140
35.0% identity (65.5% similar) in 1053 aa overlap (263-1263: 311-1347)

| | |
|---|---|
| PK1 | ERDISTEIYQAGSTPCNGVEG (SEQ ID NO: 2) |
| COV1 | ERDISNVPFSPDGKPCTP-PA (SEQ ID NO: 70) 43.8% identity (68.8% similar) |
| | in 16 aa overlap (1-16: 1-16) |
| MERS | TEVPQLVNANQYSPCVSIVP (SEQ ID NO: 71) 33.3% identity (83.3% similar) |
| | in 6 aa overlap (14-19; 13-18) |
| PK2 | IRGDEVRQIAPGQTGKIA (SEQ ID NO: 3) |
| COV1 | VKGDDVRQIAPGQTGVIA (SEQ ID NO: 72) 77.8% identity (94.4% similar) |
| | in 18 aa overlap (1-18: 1-18) |
| MERS | YPLSMKSDLSVSSAGPIS (SEQ ID NO: 73) 0% |
| PK3 | QFGRDIADTTDAVRDPQTLEI (SEQ ID NO: 4) |
| COV1 | QFGRDVSDFTDSVRDPKTSEI (SEQ ID NO: 74) 71.4% identity (90.5% similar) |
| | in 21 aa overlap (1-21: 1-21) |
| MERS | RFVYDAYQNLVGYYSDDGNY (SEQ ID NO: 75) 40.0% identity (80.0% similar) |
| | in 5 aa overlap (11-15: 5-9) |
| PK4 | EQDKNTQEVFAQVKQIYK (SEQ ID NO: 5) |
| COV1 | EQDRNTREVFAQVKOMYK (SEQ ID NO: 76) 83.3% identity (100.0% similar) |
| | in 18 aa overlap (1-18: 1-18) |
| MERS | RQDDSVRNLFASVKSSQS (SEQ ID NO: 77) 42.9% identity (92.9% similar) |
| | in 14 aa overlap (1-14: 1-14) |
| PK5 | DSFKEELDKYFKNHTSPD (SEQ ID NO: 6) |
| COV1 | DSFKEELDKYFKNHTSPD (SEQ ID NO: 78) 100% identity |
| MERS | IDFQDELDEFFKNVSTSI (SEQ ID NO: 79) 58.3% identity (100.0% similar) |
| | in 12 aa overlap (2-13: 2-13) |
| PK7 | GFNCYFPLQSYGFQPTNG (SEQ ID NO: 7) |
| COV1 | ALNCYWPLNDYGFYTTTG (SEQ ID NO: 80) 55.6% identity (88.9% similar) |
| | in 18 aa overlap (1-18: 1-18) |
| MERS | PSTVWEDGDYYRKOLSPLEGGGWLVASG (SEQ ID NO: 81) |
| PK8 | YNSASFSTFKCYGVSPTKINDLCF (SEQ ID NO: 8) |
| COV1 | YNSTFFSTFKCYGVSATKINDLCF (SEQ ID NO: 82) 87.5% identity (91.7% |
| | similar) in 24 aa overlap (1-24: 1-24) |
| MERS | LSLFSVNDFTCSQISPAAIASNCY (SEQ ID NO: 83) 30.0% identity (60.0% |
| | similar) in 20 aa overlap (5-24: 5-24) |

Accordingly, in one aspect, disclosed herein are methods of treating, inhibiting, reducing, decreasing, ameliorating, and/or preventing a coronavirus infection (such as, for example, an infection with an avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, or middle east respiratory syndrome (MERS) coronavirus (CoV) (MERS-CoV), including, but not limited to the B1.351 variant, B.1.1.7 variant, and P.1 variant) in a subject comprising administering to the subject any of the peptides, antibodies, or compositions of any preceding aspect or any combination thereof.

In one aspect, disclosed herein are method of treating, inhibiting, reducing, decreasing, ameliorating, and/or preventing a coronavirus infection in a subject comprising administering to the subject one or more chimeric peptides comprising a SARS-CoV-2 B cell epitope, a T helper (Th) epitope, and a linker joining the SARS-CoV-2 B cell epitope to the Th epitope, wherein the SARS-CoV-2 B cell epitope consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. For example, the chimeric peptide can comprise SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and/or SEQ ID NO. 23.

The disclosed methods of treatment are not limited to chimeric peptides comprising native coronavirus B cell epitopes, but can comprise coronavirus spike protein synthetic peptides and chimeric peptides comprising said synthetic peptides. Thus, in one aspect, disclosed herein are methods of treating, inhibiting, reducing, decreasing, ameliorating, and/or preventing a coronavirus infection in a subject comprising i) administering to the subject one or more coronavirus spike protein synthetic peptides wherein the coronavirus spike protein synthetic peptide comprises one or more of the sequences as set forth in SEQ ID NO: 24, SEQ ID NO: 25, SEQ ID NO: 26, SEQ ID NO: 28, SEQ ID NO: 29, or SEQ ID NO: 30; or ii) administering one or more chimeric synthetic peptides comprising a coronavirus spike protein synthetic peptides, a T helper (Th) epitope, and a linker joining the coronavirus spike protein synthetic peptides to the Th epitope; wherein the coronavirus spike protein synthetic peptide comprises SEQ ID NO: 24, SEQ ID NO: 25, SEQ ID NO: 26, SEQ ID NO: 28, SEQ ID NO: 29, or SEQ ID NO: 30. For example, the chimeric peptide can comprise SEQ ID NO: 31, SEQ ID NO: 32, SEQ ID NO: 33, SEQ ID NO: 34, SEQ ID NO: 35, SEQ ID NO: 36, or SEQ ID NO: 37.

Disclosed herein are modified coronavirus spike protein peptides. These peptides can also be used in the treatment of coronavirus infections. Thus, in one aspect, disclosed herein are methods of treating, inhibiting, reducing, decreasing, ameliorating, and/or preventing a coronavirus infection in a subject comprising i) administering to the subject one or more modified coronavirus spike protein peptides wherein the coronavirus spike protein synthetic peptide comprises one or more of the sequences as set forth in SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47; or ii) administering one or more chimeric synthetic peptides comprising a modified coronavirus spike protein peptides, a T helper (Th) epitope, and a linker joining the coronavirus spike protein synthetic peptides to the Th epitope; wherein the modified coronavirus spike protein peptide comprises SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47. For example, the chimeric peptide can comprise SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: A COVID-19 B-cell Epitope Peptide Vaccine: Eliciting Neutralizing and Protective Immunity to SARS-CoV-2

### a) Results

### (1) Identification of Potential SARS-CoV-2 Vaccine Candidates.

We have focused efforts on the development of SARS-CoV-2-S1/receptor-binding domain (RBD) and SARS-CoV-2-S2 peptide B-cell epitope vaccines. As the coronavirus S glycoprotein is surface-exposed and mediates entry into host cells, it is the main target of neutralizing antibodies (Abs) upon infection and the focus of therapeutic and vaccine design.

The selection of candidate B-cell epitopes expressed on the surface of coronavirus spike (S) envelope protein sequence was accomplished by an in-house (Peptide Companion™, 5x.com) computer-aided analysis using six correlates of antigenicity. We identified 10 potential vaccine candidates for COVID-19 and designed 5 chimeric peptide vaccine constructs incorporating two “promiscuous” T cell (MVF: measles virus fusion epitope; TT3: Tetanus toxoid epitope). 10 potential COVID-19 peptide vaccines (PK-1-PK10) have been synthesized by AAPPTEC LLC. Peptides can be purified by HPLC (>95%).

(2) In Vivo Testing in Balb/c and C57B16 Mice and Rabbits Using Two Different Adjuvants in there Different Timing Schedules.

BALB/c and C57BL6 mice have been inoculated subcutaneously with 200 μg of COVID-19 chimeric peptides emulsified in Montanide ISA720 or ISA51 (SEPPIC, Paris, France) adjuvant (1:1) and nor-MDP adjuvant (N-acetyl-glucosamine-3 yl-acetyl 1-alanyl-d-isoglutamine) and boosted twice at ONE, TWO AND THREE week intervals. This schedule can identify the best boosting schemes for enhanced immunogenicity that can easily be translated to human studies for rapid and effective deployment. PBS treated mice can be used as a negative control. Blood can be collected weekly and sera tested for antibodies by indirect ELISA. New Zealand white rabbits have been inoculated with 1.5 mg of Chimeric COVID-19 peptides emulsified in adjuvant as describe above and boosted twice at three week intervals. Blood can be collected submandibular, sera tested for Ab titers. Antibody titers can be monitored by indirect ELISA against the peptide immunogen, the peptide B cell epitope, and the recombinant protein. All experiments can be performed in accordance with the U.S. Public Health Service Policy on Humane Care and Use of Laboratory Animals and approved by the Ohio State University Institutional Animals Care and Use Committee and detailed in the accepted protocol.

Elucidating isotypes and neutralizing activities of COVID-19 polyclonal antibodies elicited by vaccination. The antibody-mediated humoral response is crucial for preventing viral infections. A subset of these antibodies, which reduce viral infectivity by binding to the surface epitopes of viral particles and thereby blocking the entry of the virus to an infected cell, are defined as neutralizing antibodies. Initially we developed a Pseudotyped lentivirus/spike protein that can be used to assess the neutralizing activity of the vaccines. We can then use a neutralization antibody assay with SARS-COV-2 (BSL3) to obtain infectivity data. These antibodies can have different isotypes and elicit a cytokine profile. Antibody isotypes (i.e., IgA, IgM, IgG1, IgG2a, IgG2b and IgG3) can be determined using the Mouse Typer Isotyping Kit (BIO-RAD). Cytokine profiles can be determined using a commercially available fluorescence-based system, MILLIPLEX MAP Mouse High Sensitivity Cytokine/Chemokine Magnetic Bead Panel, Millipore/Sigma, Burlington, MA) with an automated analyzer Luminex 200 (Luminex, Austin, TX).

Enhancing vaccine efficacy by co-vaccination with a novel PD1-Vaxx. Once the putative COVID-19 vaccine/s have been identified, it can be combined with a PD1-Vaxx to establish enhanced immunogenicity by blocking PD-1/PDL1 axis as demonstrated in U.S. application Ser. No. 16/498,929, filed on Sep. 27, 2019 and PCT Application No. PCT/US2020/051240, filed on Sep. 17, 2020, which are incorporated herein by reference in their entirety for their teachings of PD-1 and PD-L1 vaccines.

(3) Efficacy Testing in K18-hACE2 Transgenic Mice Challenged with COVID-19 SARS-CoV2 (Strain USA-WA1/2020) Virus:

The demonstration of protection from infection in animal challenge models K18-hACE2 transgenic mice available from Jackson labs with an anticipated delivery Jun. 30, 2020. We plan to breed the mice and obtain sufficient animals (initially 64 mice) in planned studies for evaluating two potential candidates in K18-hACE2-transgenic mice to determine mortality/survival challenged with SARS-CoV-2 isolate USA-WA1/2020.

### b) Methods

### (1) Peptide Synthesis, Characterization and Purification

10 COVID-19 chimeric synthetic peptides PK1 to PK10 were synthesized. The characterization (Mass SPEC) and purification were done. Briefly, peptide synthesis was performed using 9600 Milligen/Biosearch solid-phase peptide synthesizer (Millipore, Bedford, MA, USA) using Fmoc/t-Butyl chemistry and PyBOP/HOBT coupling reagents on either CLEAR amide resin or CLEAR acid resin (Peptides International, Louisville, KY, USA). All MVF/TT3 derived chimeric peptide vaccines were co-linearly synthesized with a promiscuous Th cell epitope derived from the measles virus fusion protein (MVF; residues 288-302) using a four residue linker (GPSL). Peptide mimics were acetylated using 1-Acetylimidazole (Sigma-Aldrich St. Lois, MO, USA) before cleavage. Intramolecular disulfide bonds were formed using iodine oxidation and disulfide bridge formation was further confirmed by maleimide-PEO2-biotin reaction and subsequent analysis using electrospray ionization mass spectroscopy. Peptides were cleaved from the resin using cleavage reagent R (TFA)/thioanisole/EDT/anisole (90/5/3/2), and crude peptides were purified by semi preparative (C4 or C-18 Vydac columns) reversed-phase-HPLC (Waters, Bedford, MA, USA) and characterized by MALDI (Matrix Assisted Laser Desorption Ionization mass spectroscopy at the CCIC (Campus Chemical Instrumentation Center, The Ohio State University, Columbus, OH, USA). All fractions were analyzed on analytical RP-HPLC and characterized by MALDI. RP-HPLC fractions showing same mass spectrum peak were pooled together and lyophilized.

(2) Preparation of COVID-19 Vaccine Sequences

```
(a) Sequences:
  1. TT3-PK-1: NH₂-
FNNFTVSFWLRVPKVSASHLGPSLERDISTEIYQAGSTPCNGVEG-
CONH₂ (SEQ ID NO: 14)
  2. TT3-PK-2: NH₂-
FNNFTVSFWLRVPKVSASHLGPSLIRGDEVRQIAPGQTGKIA-
CONH₂ (SEQ ID NO: 16)
  3. TT3-PK-3: NH₂-
FNNFTVSFWLRVPKVSASHLGPSLFGRDIADTTDAVRDPQTLEI-
CONH₂ (SEQ ID NO: 17)
  4. TT3-PK-4: NH₂-
FNNFTVSFWLRVPKVSASHLGPSLEQDKNTQEVFAQVKQIYK-
CONH₂ (SEQ ID NO: 18)
  5. TT3-PK-5: NH₂-
FNNFTVSFWLRVPKVSASHLGPSLDSFKEELDKYFKNHTSPD-
CONH₂ (SEQ ID NO: 19)
  6. MVF-PK-6: NH₂-
KLLSLIKGVIVHRLEGVEGPSLERDISTEIYQAGSTPCNGVEG-
CONH₂ (SEQ ID NO: 15)
  7. TT3-PK-7: NH₂-
FNNFTVSFWLRVPKVSASHLGPSLGFNCYFPLQSYGFQPTNG-
CONH₂ (SEQ ID NO: 20)
  8. TT3-PK-8:NH₂-
FNNFTVSFWLRVPKVSASHLGPSLYNSASFSTFKCYGVSPTKLNDLCF-
CONH₂ (SEQ ID NO: 21)
  9. TT3-PD-L1: NH₂-
FNNFTVSFWLRVPKVSASHLGPSLVTSEHELTCQAEGYPKAE-
CONH₂ (SEQ ID NO: 23)
10. TT3-PD-1: NH₂-
FNNFTVSFWLRVPKVSASHLGPSLGAISLAPKAQIKESLRAEL-
CONHz(SEQ ID NO: 22)
```

(3) Resin: Fmoc-Rink Amide MBHA Resin (Substitution 0.67 mmol/g) 500 mg

### (a) Amino Acids:

Fmoc-Ala-OH
Fmoc-Arg(Pbf)-OH
Fmoc-Asn(Trt)-OH
Fmoc-Asp(OtBu)-OH
Fmoc-Cys(Trt)-OH
Fmoc-Gln(Trt)-OH
Fmoc-Glu(OtBu)-OH
Fmoc-Gly-OH
Fmoc-His(Trt)-OH
Fmoc-Ile-OH
Fmoc-Leu-OH
Fmoc-Lys(Boc)-OH
Fmoc-Phe-OH
Fmoc-Pro-OH
Fmoc-Ser(tBu)-OH
Fmoc-Thr(tBu)-OH
Fmoc-Trp(Boc)-OH
Fmoc-Tyr(tBu)-OH
Fmoc-Val-OH

### (b) Coupling Reagents:

Diisopropylcarbodiimide (DIC)
Oxyma

### (c) Resin Swelling Procedure

300 mg of Fmoc-Rink Amide MBHA Resin (substitution 0.67 mmol/g, 0.20 mmol) was added to 5 ml of N,N-dimethylformamide (DMF). The mixture was shaken at room temperature for 15 minutes, then the solvent was drained. Washed the resin with 5 ml solvent (DMF) mixed 1 min.

### (d) Fmoc Deprotection Procedure

3 ml of 20% piperidine in DMF containing 0.5% DBU was added and the resin was shaken at room temperature for 5 minutes. The resin was filtered and another 3 ml portion of 20% piperidine in DMF containing 0.5% DBU was added. The mixture was shaken at room temperature for 15 minutes. The resin was filtered, then washed 5×3 ml DMF(0.5%, HOBt).

### (e) Amino Acid Coupling Procedure

5 equivalents (1.00 mmol) of Fmoc-amino acid and 5 equivalents of Oxyma (142 mg) were dissolved in 5 ml of DMF. 7.5 equivalents of DIC (568 mg, 0.70 ml) were added to the solution. The activated solution was added to the resin and was mixed for 5 min at room temperature and ramp from room temperature to 65° C. in 7 min and follow mixing for 8 min (total of 15 minutes), except for Cys and His residues. Cys and His residues are coupled at room temperature for 60 minutes and second coupling 45 minutes. The resin was filtered, then washed with DMF (3×5 ml).

### (f) Final Resin Washing and Drying

After the Fmoc group was removed from the N-terminal residue, the resin was washed with DMF (3×5 ml) and DCM (3×5 ml) and shrunk in ethanol (3×5 ml). The resin was dried to constant weight in a vacuum oven.

### (g) Cleavage Conditions

Cleavage Cocktail: Trifluoroacetic acid (TFA)/Triisopropylsilane (TIS)/Thioanisole/Water 85:5:5:5

20 ml of the cleavage cocktail were added to the peptide-resin. The mixture was mixed at room temperature for 3.0 hours. The resin was filtered and washed with 2 ml of TFA. The TFA was added to the filtrate and 100 ml of cold ethyl ether were added to precipitate the peptide. The precipitate was centrifuged to collect the peptide. The peptide was washed (3×20 ml) with cold ethyl ether.

### (h) HPLC Analysis

HPLC Solvents
   A: Water+0.1% TFA
   B: Acetonitrile+0.1% TFA
Gradient: 20-70% B, 20 minutes
Flow Rate: 1.0 ml/min
Detection Wavelength: 220 nm
Column: Spirit C18 Peptide, 150×46 mm, 5 μm particle size, 120 Å pore size

### (4) Animal Immunization.

### (a) Initial Peptide Vaccine Screen by Using C57BL/6J Wild Type Mice

C57BL/6J wild type female mice were used for initial screen the peptide vaccines. Mice with 6-8 weeks old were purchased from Charles River Laboratories. Ten mice per group, each group of mice were immunized with TT3 linked peptide vaccine, PK1, PK2, PK3, PK4, PK5, PK7 and PK8. MVF linked peptide PK6 (the same basic sequence with PK1) was used on BALB/c. The mice were taken 3 times with 100 μg/dose 1:1 mixed with ISA 720 as adjuvant with 3 weeks intervals that named as 1Y, 2Y and 3Y. The mice sera can be collected every weekly after secondary immunization, which are 2Y, 2Y+1, 2Y+2, 3Y, 3Y+1 and 3Y+2 ect. as indicated. The number after 1Y, 2Y or 3Y indicate the week after the immunization. The immunogenicity were detected by ELISA to confirm the immune response of mice. After the third immunization (3Y+3) all the mice were terminated for the final bleed.

### (b) Initial Peptide Vaccine Screen by Using New Zealand Rabbit

Female New Zealand white rabbits were purchased from Charles River Laboratories. All the designed peptide vaccine was immunized to each individual rabbit (2 kg, about 10 weeks old). The rabbit was taken 1 mg of each peptide vaccine mixed equal volume with ISA 720 as adjuvant. The rabbits were immunized three times with three weeks intervals and the sera were collected as indicate to perform immunogenicity analysis to check the immune response. After the third immunization (3Y+3) all the rabbits were terminated for the final bleed.

### (c) K18-hACE2 Transgenic Mice for SARS-CoV2 Live Virus Study

Male K18-hACE2 transgenic mice were purchased from JAX lab. We mate the male transgenic mice with C57BL/6J wild type female mice, the tissues from offspring's were sent to TransnetYX® to test the genotyping of the pups. K18-hACE2 positive mice were kept for further study. When the mice were at least 6-8 weeks, and the body weight at least over 15 gram we started to immunize the mice as the groups as indicated, each group with at least 10 mice. We immunize the mice with 3 weeks interval and 3 weeks apart. After the first immunization (1Y), the mice can receive two addition boost at 2Y and 3Y. Two weeks after the last immunization (3Y), four mice from each group of immunized mice can be sacrificed, bleed can be collected for further detection. All the left mice, 10 mice each group (5 male and 5 female) can be sent to BSL3 facility to challenge with live SARS-CoV2 virus to monitor the efficiency of vaccine. PBS group as control.

K18-hACE2 transgenic mice can be purchased from JAX and bred and maintained by ourselves. Mouse genotyping were performed by using Mouse Genotyping Kit (Cell Biologics® Cat #CB6930-500). PCR primers (5′-3′) are as followings: for internal control: forward: 5′-CTCCCAACCCCAGAGGTAGT-3′ (SEQ ID NO: 58), reverse: 5′-AGACCCCAGATCCAGAAAGG-3′ (SEQ ID NO: 59); for hACE2: forward: 5′-CCTGGCTGAAAGACCAGAAC-3′ (SEQ ID NO: 60), reverse: 5′-TCAAATTAGCCACTCGCACA-3′ (SEQ ID NO: 61), which products length are 320 bp and 249 bp, respectively. PCR thermal conditions are:

| Steps | Temperature | Duration | | Repetitions |
|---|---|---|---|---|
| 1 | 94° C. | 5 | min | 1X |
| 2 | 94° C. | 15 | s | 10X(decrease |
| 3 | 65° C. | 30 | s | 0.5° C./cycle) |
| 4 | 72° C. | 40 | s | |
| 5 | 94° C. | 15 | s | 30X cycles |
| 6 | 60° C. | 30 | s | |
| 7 | 72° C. | 40 | s | |
| 8 | 72° C. | 5 | min | 1X |

| | | | |
|---|---|---|---|
| 9 | 15° C. | forever | 1X |

PCR products were used to run gel electrophoresis and followed by using UV light with camera to check PCR bands to determine positive transgenic mice.

### (5) ELISA Immunogenicity Assay

Immunogenicity of mice or rabbits immunized with B-cell epitopes were evaluated by ELISA. Briefly, 96-well plate was coated with 100 μl of peptide the same as mice or rabbits received of 200 μg in PBS as antigen (PK1, PK2, PK3, PK4, PK5, PK7 and PK8) overnight at 4 degree. Nonspecific binding sites were blocked for 1 h with 200 μl PBS 1% BSA, and plate was washed with washing buffer (WB). Vaccine antibodies in blocking buffer were added to antigen-coated plate in duplicate wells, serially diluted 1:2 in blocking buffer, and incubated for 2 h at room temperature. After washing the plate, 100 μl of 1:500 goat anti-mouse or goat anti-rabbit IgG conjugated to horseradish peroxidase (Pierce) were added to each well and incubated for 1 h. After washing, the antibody was detected using 50 ml of 0.15% H₂O₂ in 24 mM citric acid and 5 mM sodium phosphate buffer (pH5.2) with 0.5 mg/ml 2, 20-aminobis (3-ethylbenzthiazole-6-sulfonic acid) as the chromophore. Color development proceeded for 10 min, and the reaction was stopped with 25 μl of 1% SDS. Absorbance was read at 415 nm using a BioRad Benchmark ELISA plate reader (Hercules, CA).

### (6) Antibody Isotyping Assay

The experiment assay was carrying out by following the manufacturer instruction and lab protocol. Briefly mouse antibody isotypes (i.e. IgA, IgM, IgG1, IgG2a, IgG2b, and IgG3) were determined using the Mouse Typer isotyping Kit (BIO-RAD Cat. #172-2055). Briefly, wells of a 96-well assay plate (COSTAR, REF #2797) were coated with 100 μl of 2 μg/ml peptide antigen in ddH₂O, and incubated at 4° C. overnight. The plate was washed with washing buffer (0.05% tween-20 and 1% horse sera in PBS). The plate was blocked with 1% BSA in PBS at room temperature for 1 h. 100 μl of diluted sera was added to each well. Dilutions of each sera samples were determined by the ELISA titers shown in absorbance of at least 0.4 or higher after subtracting the background, value around 1.0 are better based on your experimental condition. After washing the wells, 100 μl ready to use rabbit anti-mouse subclasses antibodies were added to each well respectively and incubated at room temperature for 2 h. The wells were washed again, 100 μl (1/3000 dilution of goat anti-rabbit conjugated to HRP antibody (BIO-RAD Cat. #172-1019)) was added to each well and incubated for 1 h at room temperature in dark. The plate received a final wash and 50 μl prepared substrate solution was added to each well (Bio Rad Cat. #1721064). The reaction was stopped with 25 μl 5% SDS stopping buffer. Absorbance at 415 nm was determined using a plate reader.

### (7) Recombinant Protein Activity Assay

For the detection of antibody reactivity with SARS or SARS-CoV2 related recombinant protein, the recombinant protein in 100 μl of PBS with 1 ug protein was coated on the 96 wells as antigen. The following procedures were performed as the description of ELISA.

### (8) Cell Culture

Vero E6 (ATCC CCL-81) cells, HEK 293 cells and hACE2-HEK293T (from BEI resource) cells were cultured at 37° C. with 5% C02 in Dulbecco's Modified Eagle medium (DMEM) supplemented with 10% fetal bovine serum (FBS).

### (9) Pseudotype SARS-CoV2 Virus Preparation

Plasmids of pcDNA3.1_SARS2_spike_del19, pUltrananoluc and psPAX2 were co-transfected into HEK 293 cells and incubate in the cell culture incubator. The following day the media was replaced to fresh primary growth media and continue to incubate the cell till the next day. Two days after the transfection, the supernatant was transfer to conical tubes and centrifuge at 1500 rpm for 5 min. the supernatant was filter through 0.45 μm syringe filter tittering immediately and aliquot into 1.5 ml tube store at −80° C. for future use. Or use it.

### (10) Pseudovirus Based Neutralization Antibody Analysis

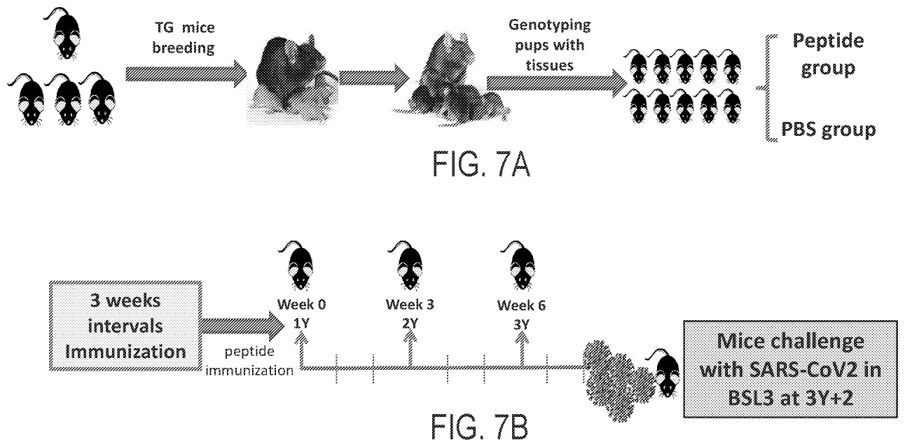

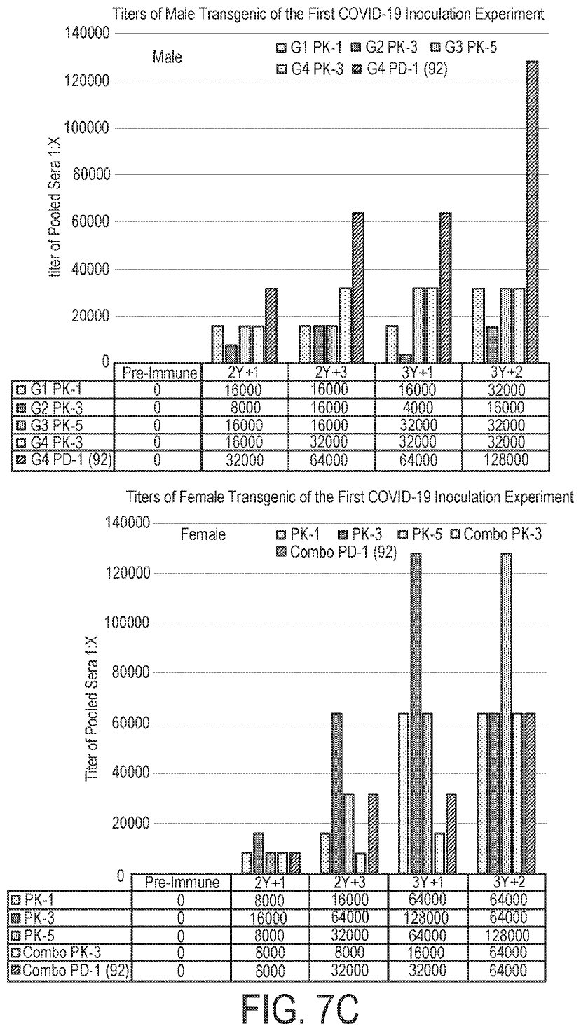

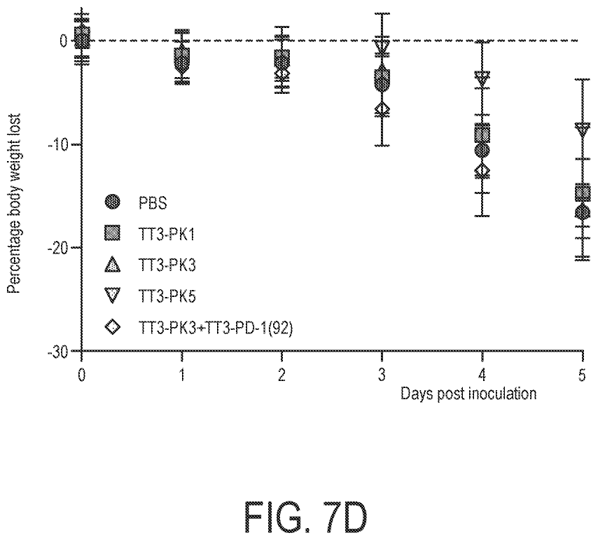

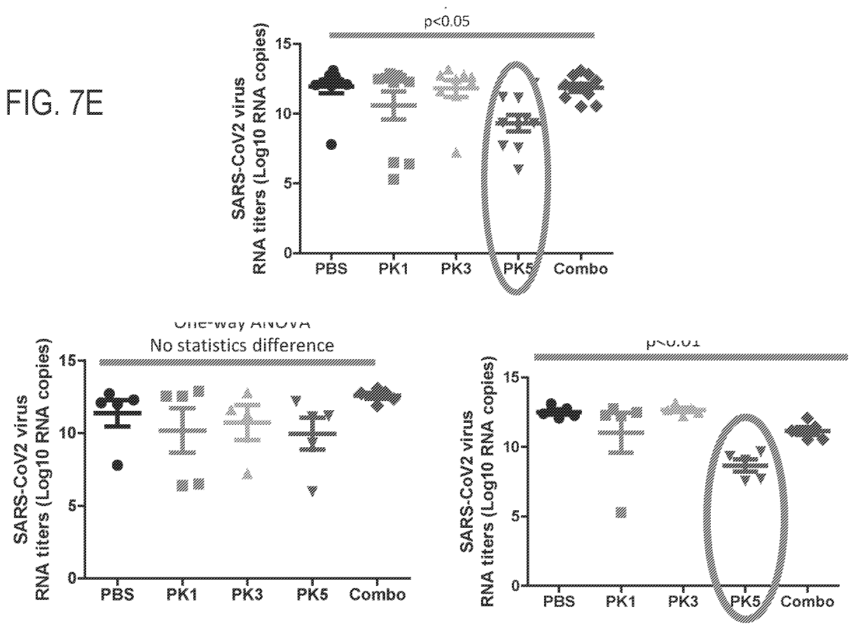

HEK 293T, hACE2-HEK 293T or Vero E6 cells can be used for neutralization assay. Here we take Vero E6 as an example. The pseudovirus was tittering by serial dilute the virus and test the relative light units by luciferase assay. For this neutralization assay we diluted the virus to around 50,000 to use (the virus relative light units might varies a lot, so the virus dilution control and no antibody neutralization assay control are necessary). One day ahead of neutralization assay, Vero E6 cells were seeded into 96 well plate, about 4,000 cells per well. On the day of assay, the virus was diluted with MEM, and sera from rabbits were diluted with MEM as designed dilution indicated in FIG. 7. Virus positive control was the virus only without mixed with any antibody, while the negative virus background control was the virus heated at 56° C. for at least 30 min or 95° C. at least 5 min. The virus was mixed with sera and incubated at 37° C. for 1 hour, then the mixture medium was transferred into 96 well plate with Vero E6 cells and incubate for 1 hour. After the incubation, the virus/sera mixture medium was replaced to fresh MEM and incubated for about another 36 hours. Luciferase assay was used to test the relative virus light unit by using Promega Luciferase assay kit as the instruction provided. And the antibody neutralization activity was analyzed.

### (11) SARS-CoV2 Virus Neutralization Analysis (BSL3).

SARS-CoV2 (strain USA-WA1/2020) was obtained from BEI resource. One day ahead of infection, we seeded 5×104 Vero E6 cells into 96-well plate per well. Mice or rabbits sera samples were heated to inactivate at 56° C. for at least 30 min or 95° C. for at least 30 min before use. Sera were serially diluted from 1:10 in culture medium. 50 μl TCID50 SARS-CoV2 were added to equal volume of the sera dilution and incubated for 1 h at 37° C. After incubation, the mixture of sera and virus were added to the prepared Vero E6 cells and keep it in the incubator for about 3 days. After incubation, cytopathic effect (CPE) of cells were checked under light microscope. The titer of neutralizing antibody were counted as the highest dilution of antibody which cause CPE in 50% of wells.

### (12) Immunofluorescence Microscopy.

Immunofluorescence microscopy can be used to detect antibody binding to cell surface with SARS-CoV2 spike protein. HEK-293T (ATCC #CRL-3216) cells seeded on glass slides are transfected with plasmids encoding SARS2-S (pcDNA3-SARS-CoV-2-S-RBD-sfGFP, Addgene plasmid #141184) by using Lipofectamine 3000 (ThermoFisher Lipofectamine™ 3000 Transfection Reagent, Cat #L3000001). Two days post transfection, cells are fixed by incubation with 2% paraformaldehyde in phosphate-buffered saline (PBS) for 30 min at room temperature and stained for nuclei with DAPI (Sigma, Catalog #D9542). Cells are then incubated with purified antibodies (PK1 to PK10) at a concentration of 10 μg/ml for 1 hour at room temperature, followed by incubation with 1:200 diluted Alexa Fluor 594 conjugated goat anti-rabbit antibodies (ThermoFisher, Cat #A-11037) for 45 min at room temperature. After staining the images can be recorded by using a confocal microscope.

### (13) Flow Cytometry-Based Receptor-Binding Inhibition Assay (BSL2)

Antibody interference of COVID-19 SARS-CoV2 S1 protein (S1N-C5255), S2 protein (S2N-C5253) and S protein RBD (SPD-C5255) binding to human ACE2 receptor on the cell surface can be measured by flow cytometry. HEK-293T cells are seeded at a density of 2×10⁵ cells per ml in a T75 flask. After reaching 70-80% confluency, cells are transfected with an expression plasmid pcDNA3-sACE2(WT)-sfGFP (Addgene plasmid 145171) encoding hACE2 with GFP marker by using Lipofectamine 3000 (ThermoFisher Lipofectamine™ 3000 Transfection Reagent, Cat #L3000001). Two days post transfection, cells are dissociated by cell dissociation solution (Sigma-aldrich, Merck KGaA; Catalog #C5914). Then 2.5 μg/ml of Fc tagged SARS-CoV2-S1, SARS-CoV2-S2 and SARS-CoV2-S RBD are pre-incubated with PK1 to PK6 antibodies sera for 1 hour on ice and subjected to flow cytometry. Single-cell suspensions in FACS buffer are centrifuged at 400 g for 10 min. Cells were subsequently incubated with SARS-CoV2-S1, SARS-CoV2-S2, SARS-CoV2-S RBD and PK1 to PK10 antibodies sera mixture for 1 hour on ice, followed by incubation with 1:200 diluted Alexa Fluor 594 conjugated goat anti-rabbit antibodies (ThermoFisher, Cat #A-11037) for 45 min at room temperature. Cells are subjected to flow cytometric analysis with a Flow Cytometer (Beckman Coulter). The results are analyzed by FlowJo (version 10). FSC/SSC gates are used to select mononuclear cells. Control antibody staining are used to define positive/negative cell populations.

(14) Animal Challenge with SARS-CoV2 Virus.

K18-hACE2 transgenic mice can be challenged with SARS-CoV2 virus (strain USA-WA1/2020 or some other new mutation strains) in this project. The mice were immunized as indicated above. After the last immunization, the mice can be transferred in to BSL3 facility and prepare to challenge with live SARS-CoV2 virus. The mice were anesthetized with Ketamine/xylazine cocktail (100 mg/ml each) 0.1 ml/20 g per mouse. Mice were intranasally inoculated (i.n.) challenged with prepared COVID-19 SARS-CoV2 (strain USA-WA1/2020) virus about 10⁵ TCID50 in 40 μl medium. The same volume of PBS was used as control. Mice body weights and healthy condition were monitored daily for the following at least a week after challenge.

### (15) Lung Tissue Histology Analysis.

10% of neutral buffered formalin was used to intratracheally fill the lungs of mice before removed. The tissues were keep at room temperature for 24 h and then embedded in paraffin. Lung specimen's sections of 5 μm were prepared and then can be stained with standard hematoxylin-eosin staining (H&E) methods.

### (16) Measurement of Viral RNA in Mice Lung Tissues

The mice were sacrificed in the ABSL3 and lung tissues were collected for further testing the SARS-CoV2 virus RNA loading in the tissues by using RT-PCR. In brief, the same amount of lung tissue was collected by using a 2 mm biopsy tissue punch from the top right part of lung. The samples were placed into 1.5 ml microcentrifuge tube with 700 μl RNAlater® (Milliporesigma) and frozen before further processing. The tissues were homogenized using the Tissuelyser II with 5 mm beads (Qiagen). The homogenates were centrifuged at 12,000 g for 1 minute and the supernatant was placed in another 1.5 microcentrifuge tube. The RNAs were extracted from 140 μl supernatant using the QIAmp Viral RNA mini kit (Qiagen). 2019-nCoV_N_Positive Control plasmid and 2019 nCoV RUO kit (IDT) were used to construct the standard curve on a Realplex master cycler (Eppendorf). The limit of detection established was 4.21 log 10 genomic copies per ml. Real time RT-PCR was conducted on experimental samples using the 2019 nCoV RUO kit and Taqman™ Fast Virus 1 step Master Mix (Thermofisher).

### (17) Statistical Analysis

For statistical analysis all data values are showed as means f standard deviation. The analysis was performed by GraphPad Prism 8.1.2 (GraphPad Software, Inc.). One-way analysis of variance (one-way ANOVA) and followed by the Tukey's multiple comparisons test were used to compare data in multiple groups or data between groups in multiple groups. And the two-way ANOVA was used to analysis the whole curves comparison. P value or adjusted p value less than 0.05 was accepted as statistically significant different.

### 2. Example 2: Peptide Based Novel SARS-CoV2 Vaccine in K18 Human Angiotensin-Converting Enzyme 2 Transgenic Mice Model

### a) Results

### (1) Design and Engineering Synthesis SARS-CoV2 Peptide Vaccines

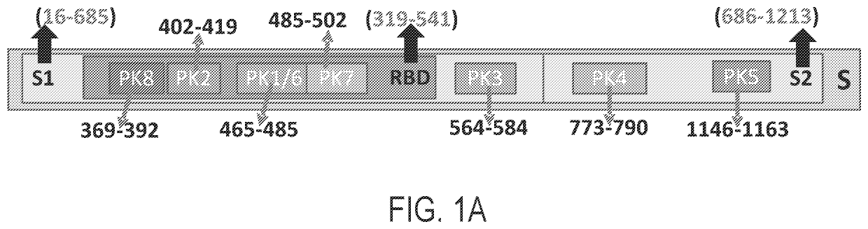

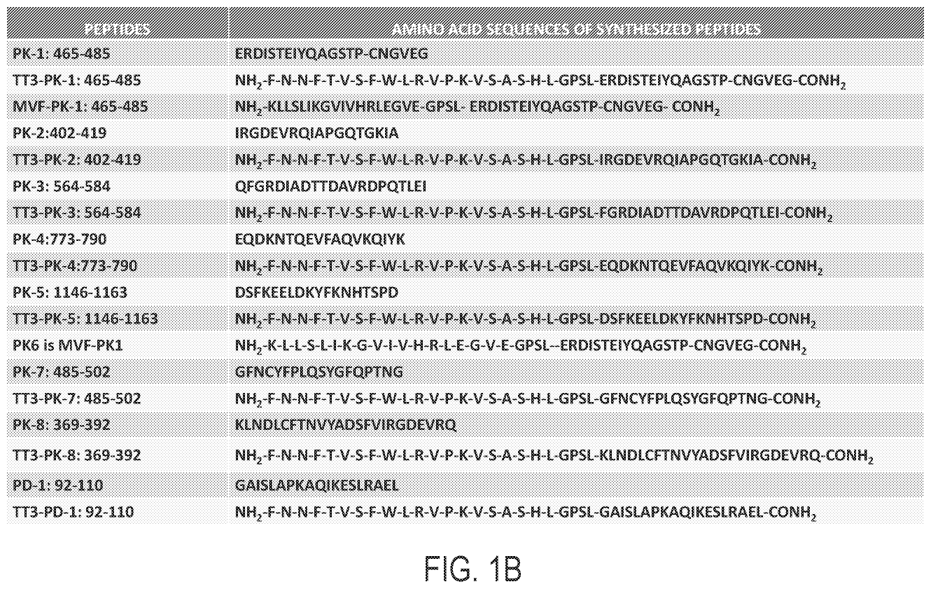

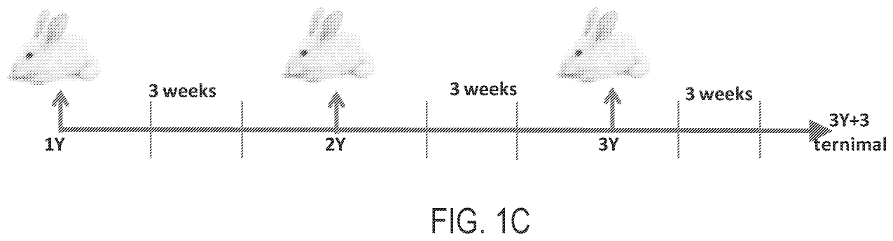

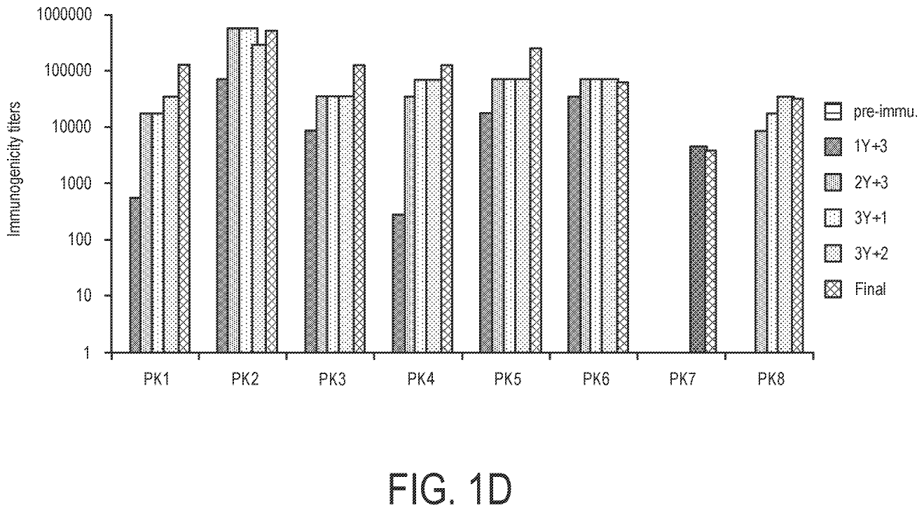

We designed 6 peptide vaccines targeted to the SARS-CoV2 spike protein (FIG. 1A). PK1/6 (465-485), PK2 (402-419) and PK3 (564-584) are located in the S1 region. PK1/6 and PK2 were designed aimed to RBD. Peptide vaccines PK4 and PK5 are located in the S2 part of the SARS-CoV2 spike protein. The peptides were linked with TT3-GPSL or MVF-GPSL based on the immunization targets. Both TT3-GPSL and MVF-GPSL linked peptide vaccines can be used on rabbits and BALB/c mice. While TT3-GPSL linked vaccines are specific for C57BL/6J background mouse instead of MVF-GPSL vaccine (FIG. 1B).

The vaccines were initially immunized on New Zealand white rabbits, about 2 kg 10 weeks old, to test the immunogenicity of each epitopes. The rabbits were immunized three times, each of individual rabbit was received two additional boosts after the primary immunization (1Y) (FIG. 1C). The final bleeds were collected three weeks after the last injection. And the immunogenicity of each epitopes was tested by ELISA (FIG. 1D). And all the rabbits were generated high titer antibodies against each epitope vaccine. Especially, PK1, PK2, PK3, PK4, and PK5 are all over 1 to 100 thousands in the terminal bleed.

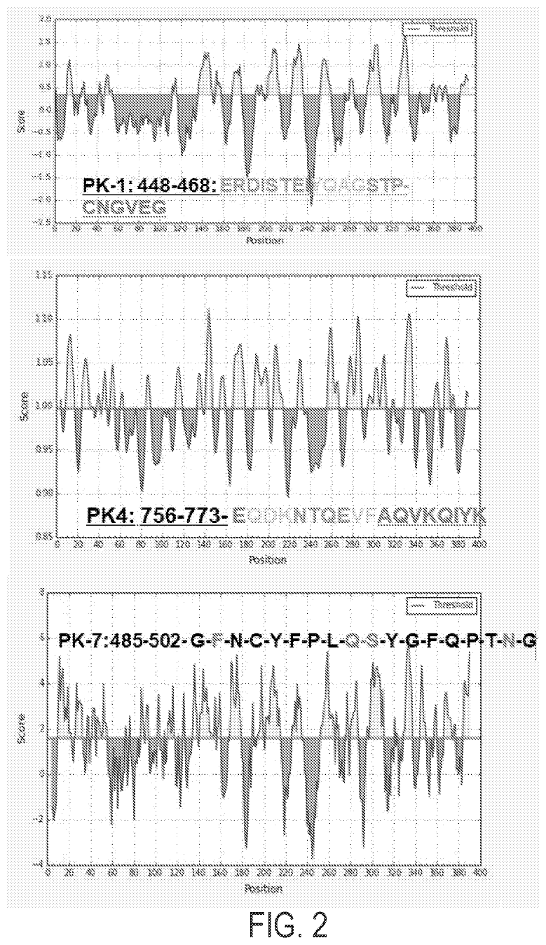

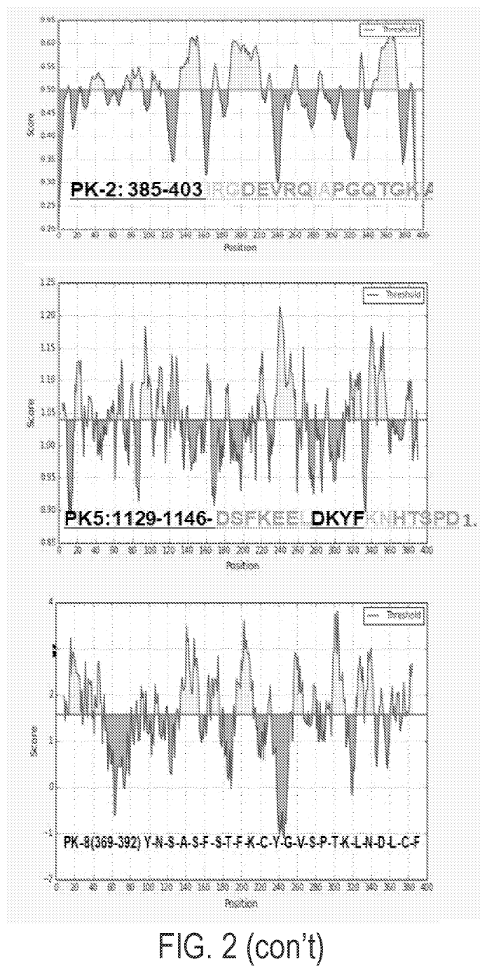

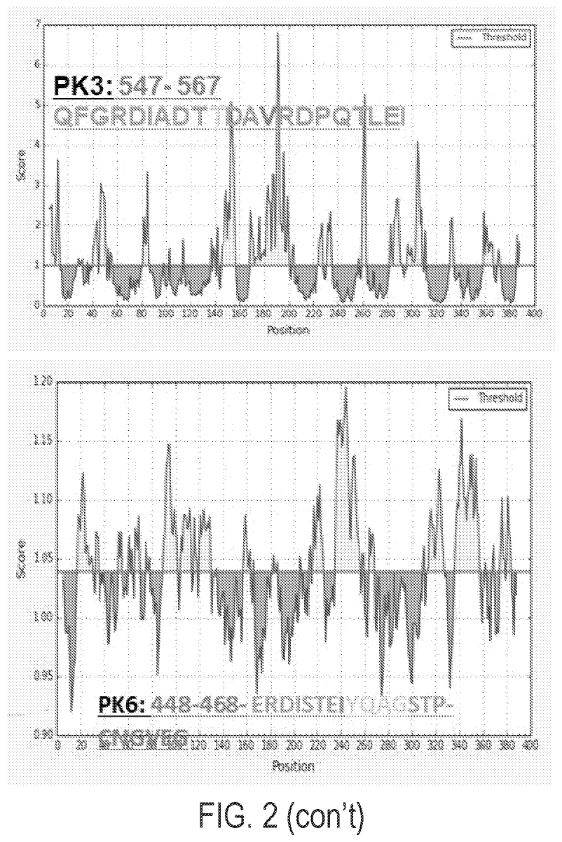

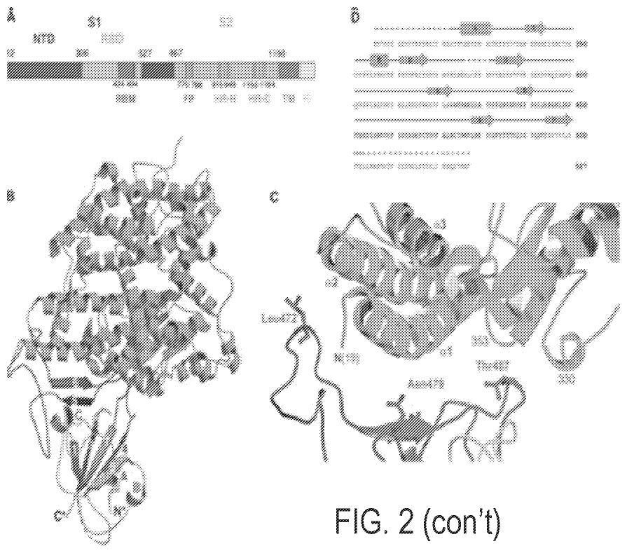

To confirm the determination of B cell the antigenicity profile of each of the B cell epitopes was obtained (FIG. 2).

### (2) Animal Immunization and Immune Response of Peptide Vaccines

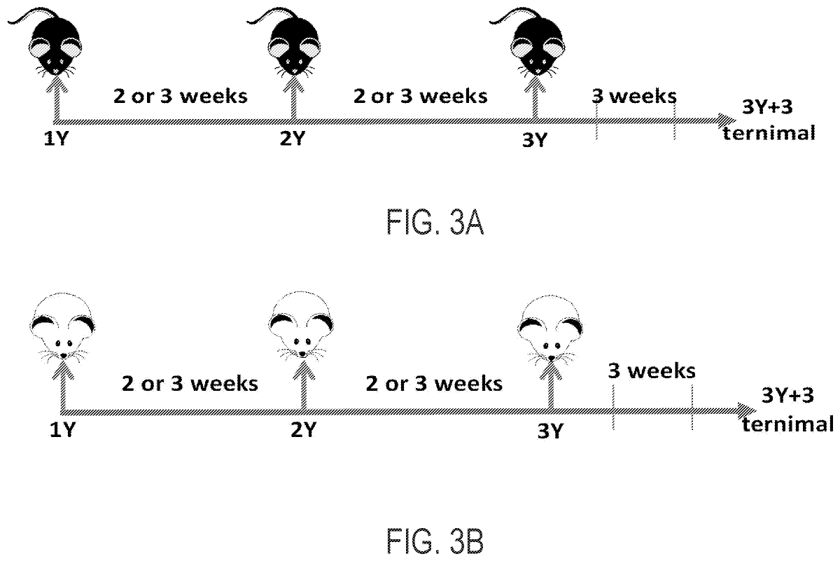

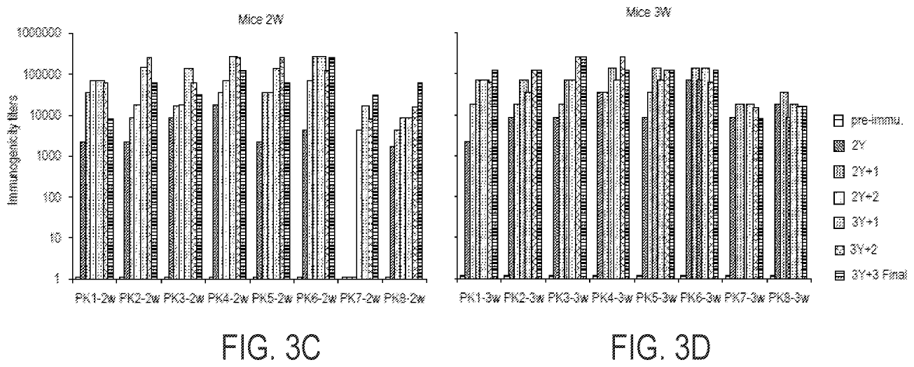

We initially immunized the peptide vaccines on wild type C57BL/6J mice and BALB/c mice. The 6-8 weeks old C57BL/6J mice (5 mice per group) were immunized with TT3-PK1, TT3-PK2, TT3-PK3, TT3-PK4 and TT3-PK5 with 2 weeks or 3 weeks intervals. While the 6-8 weeks old BALB/c mice (5 mice per group) were immunized with PK6, which is MVF-PK1 with the same procedure. The mice were immunized 3 times with 2 or 3 weeks intervals, and 3 weeks after the last immunization (3Y+3) the mice were terminated for the final bleeds (FIGS. 3A and 3B). The immunogenicity were tested by ELISA to determine the antibodies titers against its antigen as indicated (FIGS. 3C and 3D). The antibody titers were boosted during the immunization period, and all the peptide vaccines show high antibody titers except some mice immunized with 2 weeks intervals seems with slightly lower antibody titers. So the 3 weeks interval immunization strategy were used for the further study.

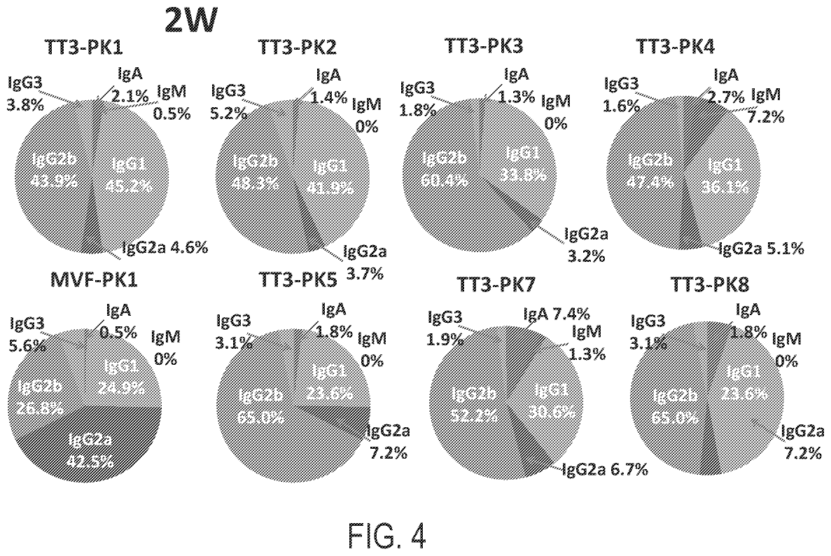

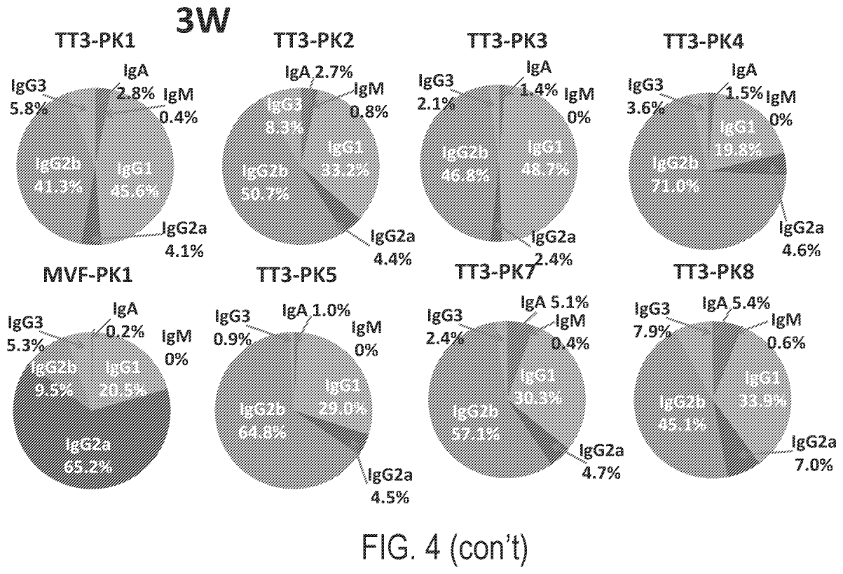

The antibodies isotypes were analyzed, the majority of IgG2b range from 41.3% to 71.0% and followed by IgG1 (19.8% TT3-PK4 to 48.7% TT3-PK3) from the mice immunized with TT3 linked peptide vaccines (FIG. 4). While the mice immunized with MVF-PK1 the majority of antibody isotype is IgG2a range from 42.5% to 65.2%, and followed by IgG2b (9.5% to 26.8%) and IgG1 (20.5% to 24.9%).

(3) Antibody Across Activities with SARS-CoV2 Recombinant Proteins and Pseudotype SARS-CoV2 Virus Neutralization Assay

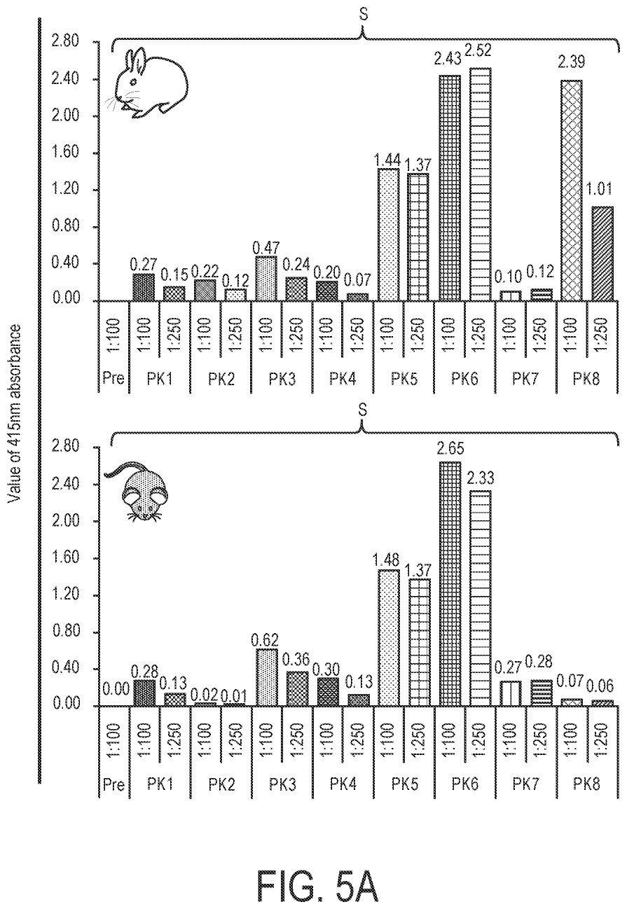

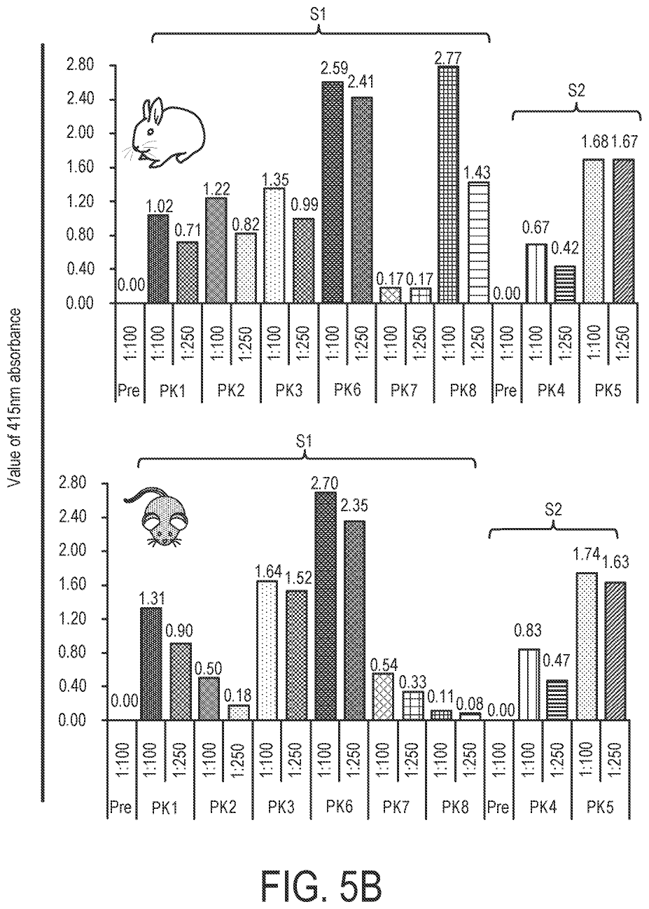

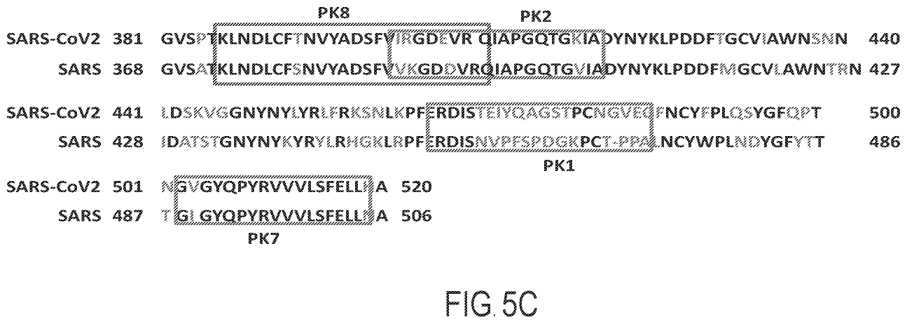

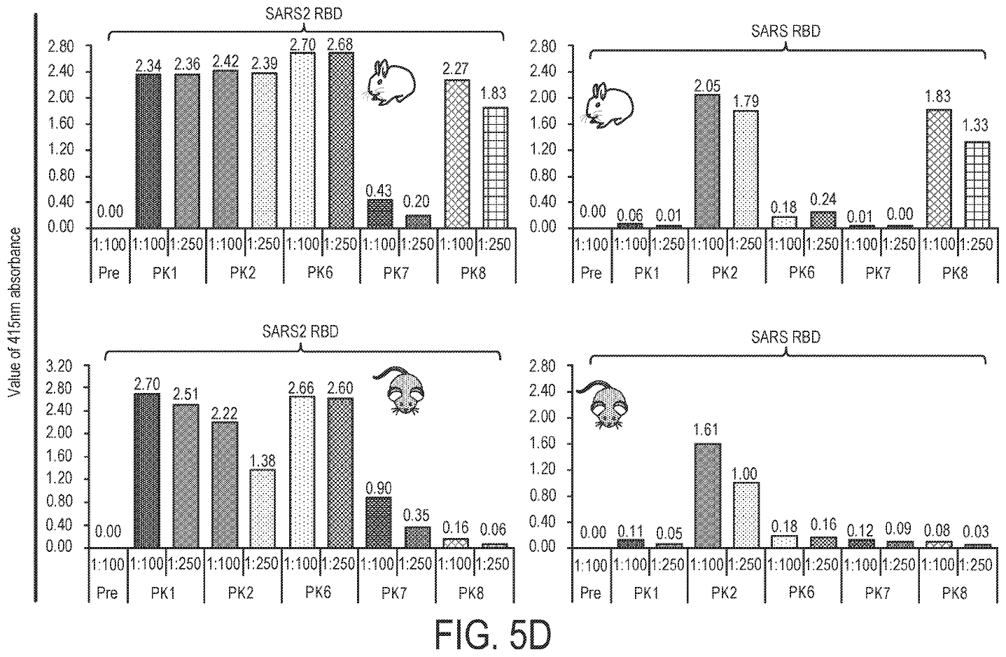

As we detected the antibodies immunogenicity from rabbits and mice, all the sera are with high binding ability against its antigen. We further analysis the antibodies cross activities with SARs-CoV2 spike recombinant protein, S1 and S2 (FIGS. 5A and 5B). Both sera from rabbits and mice which immunized with PK5 shows the highest binding with recombinant SARS-CoV2 spike protein, followed by PK3 both on rabbits and mice. And as expected, PK5 shows the higher binding ability with SARS-CoV2 S2 recombinant protein and followed by the PK3 with the highest binding with SARS-CoV2 S1 recombinant protein.

The peptide vaccines PK1 and PK2 were designed located in the SARS-CoV2 spike protein RBD region. And we know that the S protein from SARA-CoV and SARS-CoV2 with over 70% of amino acid identification. Compared SARS-CoV2 with SARS RBD, PK2 peptide located region with 14 amino acids out of 18 are same, while the sequence are quite difference between SARS-CoV2 and SARS within the PK1 located area (FIG. 5C). As indicated, both rabbits and mice immunized with PK1 and PK2 are with very high binding ability with SARS-CoV2 RBD recombinant protein, the value versus with recombinant protein activity (FIG. 5D), and the sera from PK2 immunized animals showed higher across activity with SARS RBD which was as predicted.

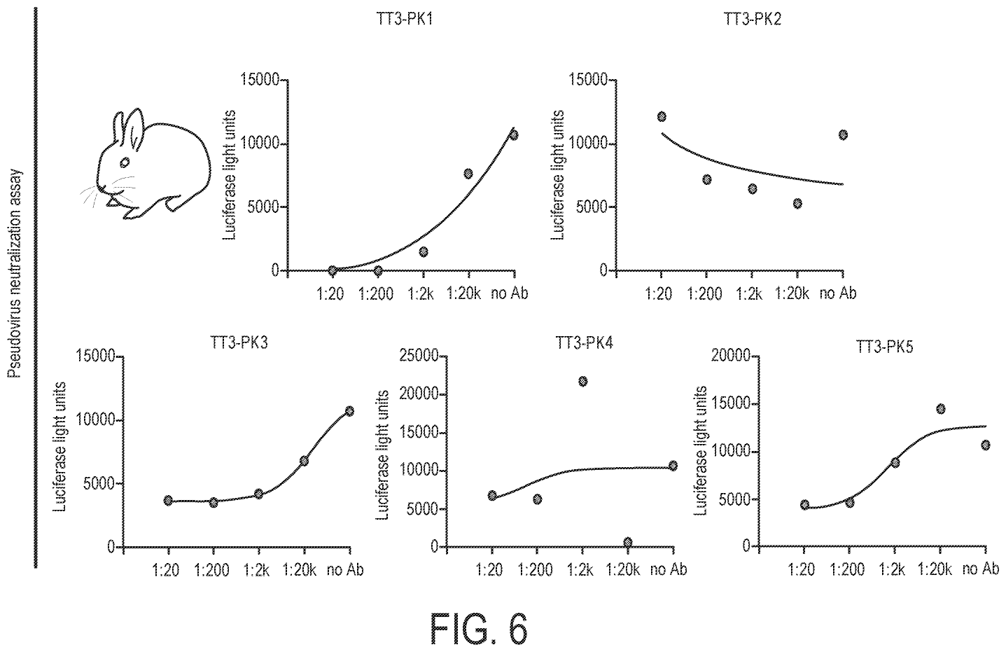

In order to confirm which peptide vaccine might work well against live SARS-CoV2 virus, pseudovirus neutralization assay were performed. Based on the results, PK5, PK3 and PK1 showed good does dose-dependent neutralization ability were selected for the further investigation (FIG. 6). While, PK2 and PK4 were put aside at the moment and some more research need to be done to confirm their neutralization ability.

Pseudotyped SARS-CoV2 virus was constructed by transfecting Vero E6 cells with lentivirus plasmid that express SARS-CoV2 spike protein. Pseudovirus was harvested for the further neutralization assay. The Y axle shows the relative luciferase light units of virus and the X axle indicates the sera dilution rate. The sera from rabbits immunized with PK1, PK3 and PK5 showed relatively good concentration dependent neutralization curves instead of PK2 and PK4 have big variation from the assay.

### (4) K18-hACE2 Transgenic Mice Immune Response to Peptide Vaccine

K18-hACE2 transgenic mice originally established by Dr. Stanley Perlman and Dr. Paul B McCray and their colleagues. Several studies have released that hACE2 transgenic mice became highly susceptible to SARS-CoV coronavirus infection. With 76% amino acid identity between the S protein from SARA-CoV and SARS-CoV2 genomic and structural homology, this support the ACE2 served as the same cell surface receptor for SARS-CoV2. After infect with SARS-CoV2, the K18-hACE2 transgenic mice showed clinical symptoms. And the SARS-CoV2 infected K18-hACE2 mice show a dose-dependent manner with death respiratory illness, moreover the subsequence of brain infection can cause death as well. K18-hACE2 (FIG. 7A) and other human ACE2 transgenic mice is a suitable animal model to investigate vaccines against current pandemic.

The male K18-hACE2 transgenic mice were purchased from JAX lab and mated with C57BL/6J female mice at OSU under ULAR policy. The off springs tissue samples were collected form ears and were sent to TRANSNETYX® to perform genotyping. The hACE positive transgenic mice were kept for further usage.

The mice were divided into 5 groups. Due to the shortage of transgenic mice, both male and female mice are mixed in each, 10 to 14 mice per group with 50% male and 50% female mice. The mice immunized with PK1, PK3, PK5 or PK3 combination vaccine, the group revived PBS was as control. The mice were received 3 times immunization with 3 weeks intervals (FIG. 7B). The mice were monitored at least three time per week and individual bleeds were collected for further analysis. Two weeks after the last immunization (3Y), the mice were taken into BSL3 facility and then inoculate with 30 μl/nostril, total 60 μl/i.n. Robust SARS-CoV2 peptide epitope vaccine immunogenicity responses were elevated in all the immunized groups as determined by ELISA (FIG. 7C). Which indicated the antibodies were boosted in both male and female immunized transgenic mice. The mice body weights were monitored daily after inoculated with SARS-CoV2 live virus (FIG. 7D). Versus with PBS control group, only the mice immunized with PK5 showed significant less body weight loss. And the SAR-CoV2 viral RNA titers in lung tissues loading results indicated that the PK5 immunized mice with much less viral RNA titers, which is significantly lower than any other groups (FIG. 7E). Due to the extremely high does SARS-CoV2 virus inoculation, 60 μl TICD₅₀ per mouse. The majority of mice in all the groups are dead or sacrificed within 7 days post inoculation. Study already indicated that the SARS-CoV2 infected K18-hACE2 mice show a dose-dependent manner, and would cause brain damage and other organs malfunction results in early death. The log 10 RNA titers normally under 7 even in PBS group with total volume as high as 50 μl per mouse. Here the results showed inoculated 60 μl SARS-CoV2 virus per mouse the final log 10 RNA titers around 12 which is much higher versus with other related researches. However, the significantly lower viral RNA titers in PK5 immunized groups indicates the vaccine has some protection effect.

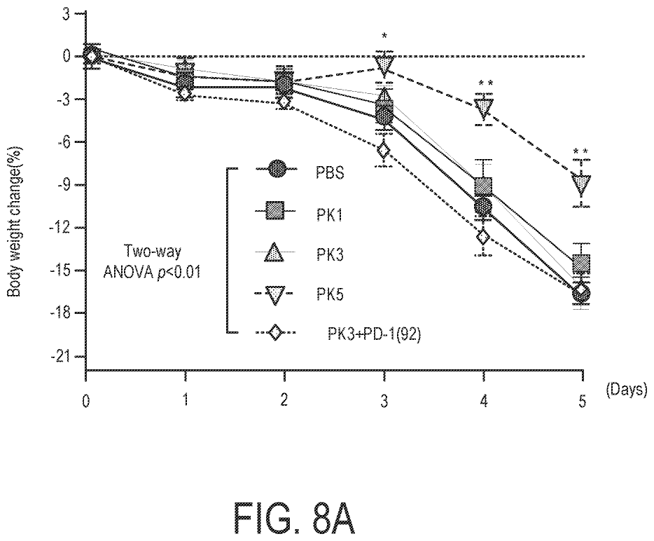

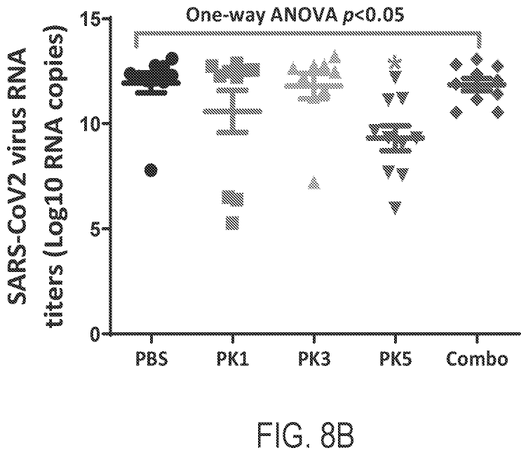

The results showed that the peptide vaccine protected mice body weight loss and viral RNA load. As shown in FIG. 8, only mice immunized with PK5 showed significant lower body weight loss (8A) and lower viral RNA loading (8B) versus PBS control. All TG mice were inoculated with 240,000 pfu/mouse SARS-CoV2 (USA-WA1-2020 strain).

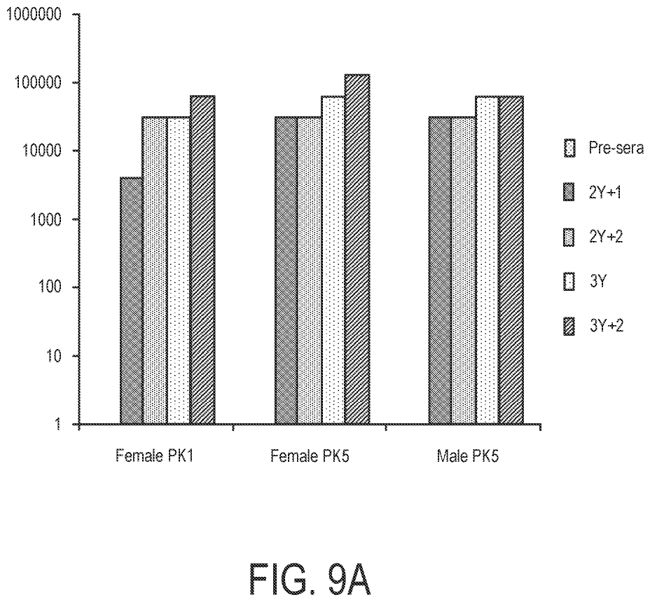

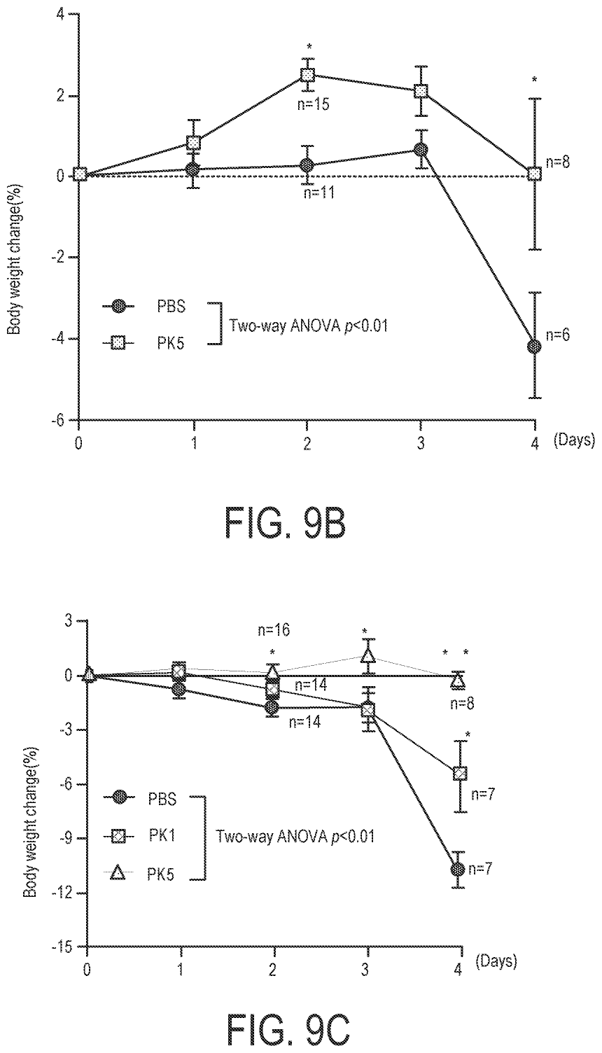

Next we investigated the ability of the transgenic mice to survive a second viral challenge. All TG mice inoculated with 40,000 pfu/mouse SARS-CoV2 (USA-WA1-2020 strain). Immunogenicity as shown in FIG. 9A were determined by ELISA. Antibodies were boosted in both male and female immunized transgenic mice. As shown in FIGS. 9B and 9C, mice body weight were monitored daily after virus challenge. 50% of mice in each group were sacrificed on day 2 post inoculation, all left over mice were sacrificed on day 4 post inoculation. Day0 body weigh of each mouse was used as base line. PK5 significantly protected mice from body weight loss.

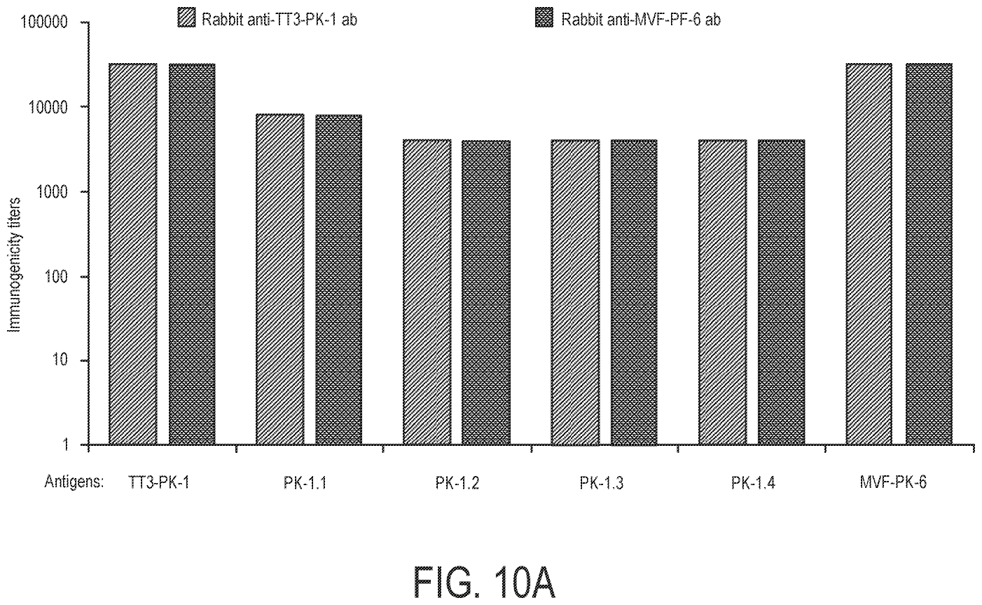

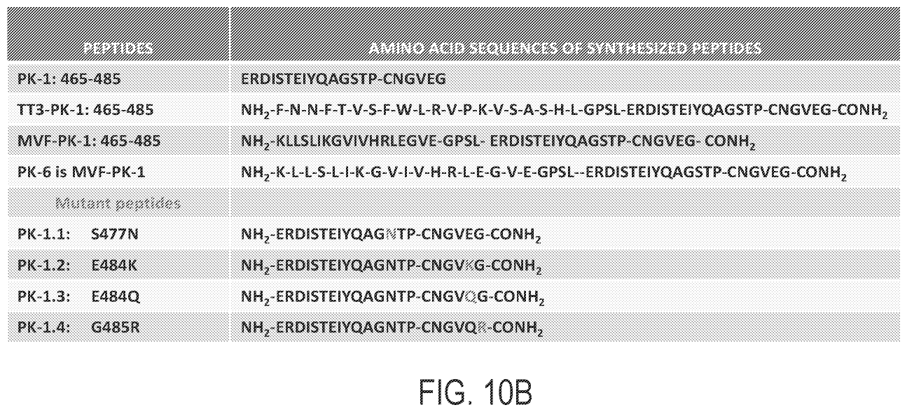

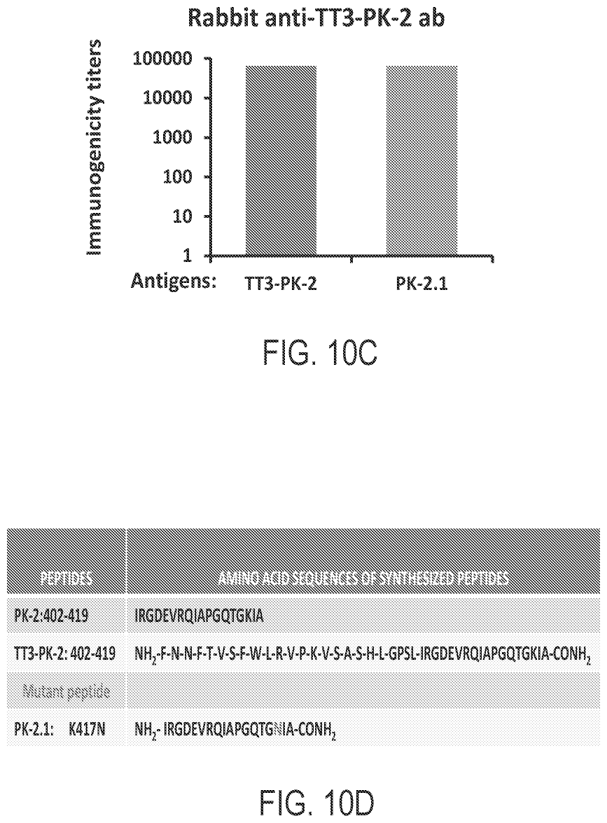

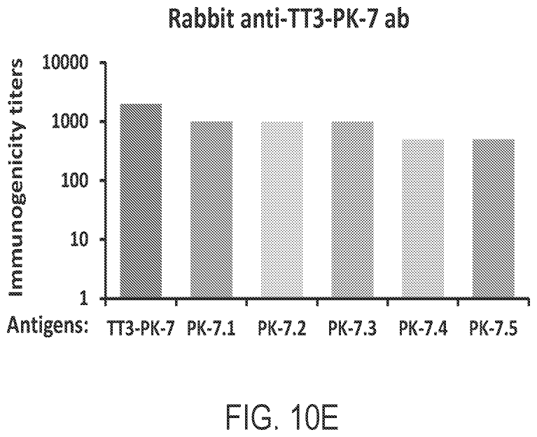

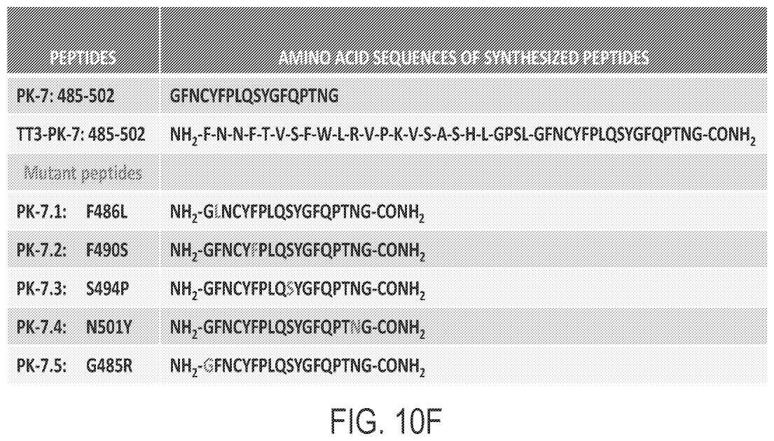

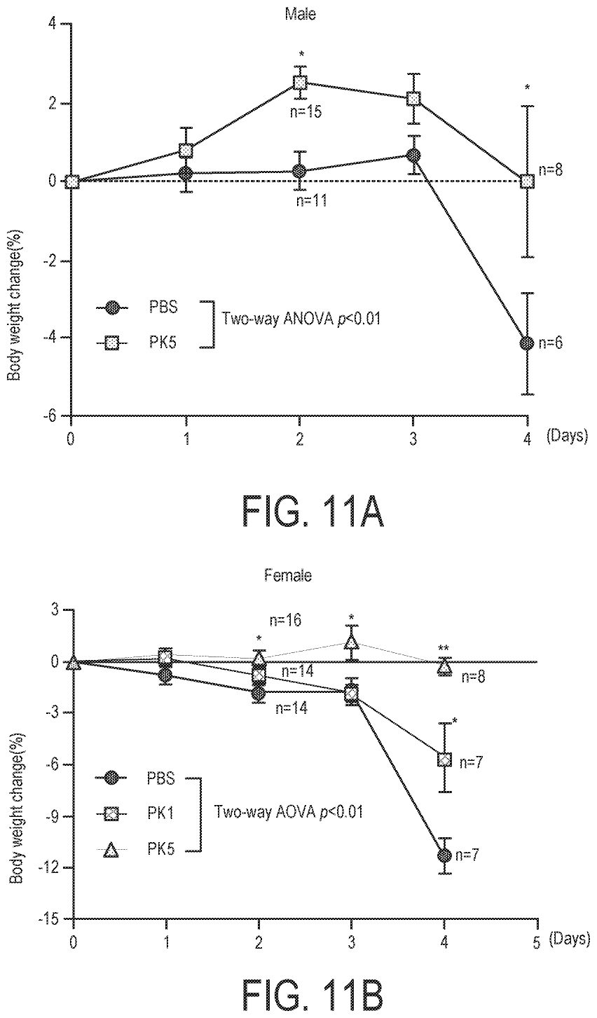

As SARS-CoV2 strain variants have emerged, we wanted to test the ability of the anti-PK-1, anti-PK-2, and anti-PK-7 antibodies to bind PK-1, PK-2, and PK-7 mutants with one or more substitutions present in mutant strains (FIG. 10A-F). Immunogenicity dropped for the anti-PK-2 antibody when mutations were present, but still showed significant binding. However, no change was observed in the ability of either the anti-PK-2 or anti-PK-7 antibodies to bind to mutated PK-1 or PK-7 epitopes showing significant cross-reactivity.

Lastly, we investigated the ability of transgenic male mice either immunized with PK5 or a control (11A) or female transgenic mice either immunized with PK1, PK5, or a control to control a viral challenge. All TG mice inoculated with 40,000 pfu/mouse SARS-CoV2 (USA-WA 1-2020 strain). The mice body weight was monitored daily after virus challenge. 7 mice in PBS group and 7 mice in PK1 and 8 mice in PK5 (7 mice in the male transgenic experiment) immunized groups were sacrificed on day 2 post inoculation, all the left over mice were sacrificed on day 4 post inoculation. Day0 body weight of each mouse was used as base line. PK5 immunized mice with significant better body weight maintain ability versus with PBS control mice. The mice in PBS group body weight drop 4.2% on average on day 4 which is significant different with PK5 group mice.

### b) Methods

### (1) Experimental Methods

All live SARS-CoV2 virus (strain of USA-WA1/2020 or other strains) related experiments were performed in the biosafety level 3 facility, Plant and Animal Agriculture Research Facility (PAAR).

### (2) Peptide Synthesis

The APPTEC Company synthesized the novel TT3-PK1, TT3-PK2, TT3-PK3, TT3-PK4, TT3-PK5 and MVF-PK6 peptide vaccines for us. We performed the further procedure to purify the crude peptides. The crude peptides were purified by semi preparative (C-4 or C-18 Vydac columns) reversed-phase-HPLC (Waters, Bedford, MA, USA) and characterized by MALDI (Matrix Assisted Laser Desorption Ionization mass spectroscopy at the CCIC (Campus Chemical Instrumentation Center, The Ohio State University, Columbus, OH, USA). All fractions were analyzed on analytical RP-HPLC and characterized by MALDI. RP-HPLC fractions showing same mass spectrum peak were pooled together and lyophilized.

(3) Animals: Rabbits, C57BL/6J Wild Type Mice and K18-hACE2 Transgenic Mice

All experiments were performed in accordance with the U.S. Public Health Service Policy on Humane Care and Use of Laboratory Animals and approved by the Ohio State University Institutional Animals Care and Use Committee and detailed in the accepted protocol. C57BL/6J wild type female mice were purchased from Charles River Laboratories Ashland, LLC, United States and K18-hACE2 transgenic mice were purchased from JAX Laboratories, LLC, United States. All animal care and use was in accordance with ULAR institutional guidelines.

### (4) Animal Immunization.

For each peptide, vaccine antibodies were raised using New Zealand female white rabbits (2 Kg/10 weeks) purchased from Charles River Laboratories (Wilmington, MA, USA). Rabbits were immunized with 1 mg of TT3 linked peptide and boosted twice at three week intervals. Both C57BL/6J wild type and K18-hACE2 transgenic mice were used in this study. And five peptide based candidates vaccines were investigated. The C57BL/6J wild type mice initially (5-6 weeks olds) were started to immunize with the peptide based vaccine to screen the peptide vaccines, which were taken 3 times with 3 weeks intervals and named as 1Y, 2Y and 3Y. The mice sera will be collected every weekly after secondary immunization, which are 2Y, 2Y+1, 2Y+2, 3Y, 3Y+1 and 3Y+2 etc., K18-hACE2 transgenic mice with C57BL/6J background were immunized with the same procedure and then transferred to ABSL3 challenged with SARS-CoV2. The vehicle Montanide ISA720™ was purchased from SEPPIC and it had an approval certificate of analyses for toxicity, emulsifying property, and sterility.

### (5) Cell Lines and SARS-CoV2 Virus Preparation

Vero E6 (ATCC CCL-1586) and hACE2-HEK293T(CSC-ACE01) cell lines were cultured at 37° C. with 5% CO₂ in Dulbecco's Modified Eagle medium (DMEM) supplemented with 10% fetal bovine serum (FBS). Vero E6 cells were used to amplification, titration and storage of COVID-19 virus of SARS-CoV2 USA-WA1/2020 strain.

### (6) Plasmid and Pseudovirus Construction

Pseudovirus were produced by co-transfect HEK-293T cells with pNL4-3.luc.RE and pcDNA3.1-SARS-CoV-S plasmid which encode SARS-CoV-2 S protein or empty vector. The pseudotyped virus were/will be in the supernatant which was/will be collected at 3-day after transfection. The supernatant was/will be filtered and centrifuged at 1500 g for 10 min and passed through 0.45 μm filter, and was/will be kept in −80° C.

### (7) Pseudovirus Based Neutralization Assay

Pseudovirus were produced by co-transfect HEK-293T cells with pNL4-3.luc.RE and pcDNA3.1-SARS-CoV-S plasmid which encode SARS-CoV-2 S protein or empty vector. The pseudotyped virus were/will be in the supernatant which was/will be collected at 3-day after transfection. The supernatant was/will be filtered and centrifuged at 1500 g for 10 min and passed through 0.45 μm filter, and was/will be kept in −80° C. The pseudovirus based neutralization antibody analysis was/will be performed as following. One day ahead of infection, we seeded 5×10⁴ hACE2-HEK293T cells into 96-well plate per well. Mice sera samples were heated to inactivate at 56° C. for 30 min before use. Sera were serially diluted from 1:10 in culture medium. 50 μl supernatant with pseudovirus were added to equal volume of the sera dilution and incubated for 1 h at 37° C. After incubation, the mixture were added to the prepared hACE2-HEK293T cells and keep it in the incubator for 2 days. The neutralization antibody titers were/will be determined by luciferase activity, which was/will be performed by standard Luciferase Assay System (Promega).

### (8) SARS-CoV2 Live Virus Neutralization Assay

One day before infection, seed 5×10⁴ Vero E6 cells per well to 96-well plate. Mice sera samples are heat inactivated at 56° C. for 30 min. Sera will be serially diluted from 1:10 in culture medium. 50 ul TCID50 COVID-19 will be added to equal volume of the serum dilution and incubated for 1 h at 37° C. Add sera and virus mixture to prepared Vero E6 cells and incubated for 2 days. After incubation, neutralization will be assessed by cytopathic effect (CPE). The neutralization endpoint was taken as the last well complete neutralization was observed, or completely prevented CPE in 50% of the wells.

(9) K18-hACE2 Mice Challenge with SARS-CoV2 Virus

K18-hACE2 transgenic mice were challenged with SARS-CoV2 virus in ABSL3. The mice at least 5-6 weeks olds were started to immunize with peptide based. Mice were transferred to ABSL3 after the last immunization 3Y. Two weeks after 3Y mice were anesthetized with Ketamine/xylazine cocktail (100 mg/ml each) 0.1 ml/20 g per mouse. Mice were intranasally inoculated (i.n.) challenged with prepared COVID-19 SARS-CoV2 (strain USA-WA1/2020) virus about 10⁵ TCLD50 in 40 μl medium. Mice body weights and healthy condition were monitored daily for at least four days and longer.

### (10) Measurement of Viral RNA in Mice Lung Tissues

The mice were sacrificed in the ABSL3 and lung tissues were collected for further testing the SARS-CoV2 virus RNA loading in the tissues by using RT-PCR. In brief, the same amount of lung tissue was collected by using a 2 mm biopsy tissue punch from the top right part of lung. The samples were placed into 1.5 ml microcentrifuge tube with 700 μl RNALATER® (Milliporesigma) and frozen before further processing. The tissues were homogenized using the Tissuelyser II with 5 mm beads (Qiagen). The homogenates were centrifuged at 12,000 g for 1 minute and the supernatant was placed in another 1.5 microcentrifuge tube. The RNAs were extracted from 140 μl supernatant using the QIAmp Viral RNA mini kit (Qiagen). 2019-nCoV_N_Positive Control plasmid and 2019 nCoV RUO kit (IDT) were used to construct the standard curve on a Realplex master cycler (Eppendorf). The limit of detection established was 4.21 log 10 genomic copies per ml. Real time RT-PCR was conducted on experimental samples using the 2019 nCoV RUO kit and Taqman™ Fast Virus 1 step Master Mix (Thermofisher

### (11) ELISA Immunogenicity Assay

The antibody titers in sera were measured. Briefly, 96-well plate was coated with 100 ul of TT3-PK1, TT3-PK2, TT3-PK3, TT3-PK4 and TT3-PK5 with 200 μg peptide in PBS overnight at 4 degree C. Nonspecific binding sites were blocked for 1 h with 200 μl PBS 1% BSA, and plate was washed with washing buffer (WB). Vaccine antibodies in blocking buffer were added to antigen-coated plate in duplicate wells, serially diluted 1:2 in blocking buffer, and incubated for 2 h at room temperature. After washing the plate, 100 μl of 1:500 goat anti-mouse IgG conjugated to horseradish peroxidase (Pierce) were added to each well and incubated for 1 h. After washing, the antibody was detected using 50 ml of 0.15% H₂O₂ in 24 mM citric acid and 5 mM sodium phosphate buffer (pH5.2) with 0.5 mg/ml 2, 20-aminobis (3-ethylbenzthiazole-6-sulfonic acid) as the chromophore. Color development proceeded for 10 min, and the reaction was stopped with 25 μl of 1% SDS. Absorbance was read at 415 nm using a BioRad Benchmark ELISA plate reader (Hercules, CA).

### (12) Recombinant Protein Activity Assay

For the detection of SARS-CoV RBD, SARS-CoV2 Spike, SARS-CoV2 S1, SARS-CoV2 S2 and SARS-CoV2 RBD recombinant protein activity, each recombinant protein in 100 μl of PBS with 1 μg protein was coated on the 96 wells as antigen. The following procedures were performed.

### (13) Antibody Isotyping Assay

The experiment assay was carrying out by following the manufacturer instruction and lab protocol. Briefly, mouse antibody isotypes (i.e. IgA, IgM, IgG1, IgG2a, IgG2b, and IgG3) were determined using the Mouse Typer isotyping Kit (BIO-RAD Cat. #172-2055). Briefly, wells of a 96-well assay plate (COSTAR, REF #2797) were coated with 100 μl of 2 μg/ml peptide antigen in ddH2O, and incubated at 4° C. overnight. The plate was washed with washing buffer (0.05% tween-20 and 1% horse sera in PBS). The plate was blocked with 1% BSA in PBS at room temperature for 1 h. 100 μl of diluted sera was added to each well. Dilutions of each sera samples were determined by the ELISA titers shown in absorbance of 0.4 or higher after subtracting the background. After washing the wells, 100 μl ready to use rabbit anti-mouse subclasses antibodies were added to each well respectively and incubated at room temperature for 2 h. The wells were washed again, 100 μl (1/3000 dilution of goat anti-rabbit conjugated to HRP antibody (BIO-RAD Cat. #172-1019)) was added to each well and incubated for 1 h at room temperature in dark. The plate received a final wash and 50 μl prepared substrate solution was added to each well (Bio Rad Cat. #1721064). The reaction was stopped with 25 μl 5% SDS stopping buffer. Absorbance at 415 nm was determined using an ELISA plate reader.

### (14) Statistical Analysis

For statistical analysis all data values are showed as means f standard deviation. The analysis was performed by GraphPad Prism 8.1.2 (GraphPad Software, Inc.). One-way analysis of variance (one-way ANOVA) and followed by the Tukey's multiple comparisons test were used to compare data in multiple groups or data between groups in multiple groups. And the two-way ANOVA was used to analysis the whole curves comparison. P value or adjusted p value less than 0.05 was accepted as statistically significant different.

E. Sequences

SEQ ID NO: 1 SARS-COV2 Spike Protein

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfcndpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkglssnf gaissvlndi lsrldkveae vqidrlitgr lqslqtyvtq qliraaeira |
| 1021 | sanlaatkms ecvlgqskrv dfcgkgyhlm sfpqsaphgy vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeqyi kwpwyiwlgf iagliaivmv timlccmtsc csclkgcesc gscckfdedd |
| 1261 | sepvlkgvkl hyt |

SEQ ID NO: 2 PK-1 (465-485)
ERDISTEIYQAGSTPCNGVEG
SEQ ID NO: 3 PK-2 (402-419)
IRGDEVRQIAPGQTGKIA
SEQ ID NO: 4 PK-3 (564-584)
QFGRDIADTTDAVRDPQTLEI
SEQ ID NO: 5 PK-4 (773-790)
EQDKNTQEVFAQVKQIYK
SEQ ID NO: 6 PK-5 (1146-1163)
DSFKEELDKYFKNHTSPD
SEQ ID NO: 7 PK-7 (485-502)
GFNCYFPLQSYGFQPTNG
SEQ ID NO: 8 PK-8 (369-392)
KLNDLCFTNVYADSFVIRGDEVRQ
SEQ ID NO: 9 PD-1 (92-110)
GAISLAPKAQIKESLRAEL
SEQ ID NO: 10 PD-L1 (130-147)
VTSEHELTCQAEGYPKAE
SEQ ID NO: 11 Measles virus fusion protein (MVF)
KLLSLIKGVIVHRLEGVE
SEQ ID NO: 12 TTE
FNNFTVSFWLRVPKVSASHLE
SEQ ID NO: 13 Linker
GPSL
SEQ ID NO: 14 TT3-PK1 (465-485)
FNNFTVSFWLRVPKVSASHLEGPSLERDISTEIYQAGSTPCNGVEG
SEQ ID NO: 15 MVF-PK1 (465-485)
KLLSLIKGVIVHRLEGVEGPSLERDISTEIYQAGSTPCNGVEG
SEQ ID NO: 16 TT3-PK2 (402-419)
FNNFTVSFWLRVPKVSASHLEGPSLIRGDEVRQIAPGQTGKIA
SEQ ID NO: 17 TT3-PK3 (564-584)
FNNFTVSFWLRVPKVSASHLEGPSLQFGRDIADTTDAVRDPQTLEI
SEQ ID NO: 18 TT3-PK4 (773-790)
FNNFTVSFWLRVPKVSASHLEGPSLEQDKNTQEVFAQVKQIYK
SEQ ID NO: 19 TT3-PK5 (485-502)
FNNFTVSFWLRVPKVSASHLEGPSLDSFKEELDKYFKNHTSPD
SEQ ID NO: 20 TT3-PK7 (773-790)
FNNFTVSFWLRVPKVSASHLEGPSLGFNCYFPLQSYGFQPTNG
SEQ ID NO: 21 TT3-PK8 (369-392)
FNNFTVSFWLRVPKVSASHLEGPSLKLNDLCFTNVYADSFVIRGDEVRQ
SEQ ID NO: 22 TT3-PD-1 (92-110)
FNNFTVSFWLRVPKVSASHLEGPSLGAISLAPKAQIKESLRAEL
SEQ ID NO: 23 TTE-PD-L1 (130-147)
FNNFTVSFWLRVPKVSASHLEGPSLVTSEHELTCQAEGYPKAE
SEQ ID NO: 24 PK-1 (465-485) D PEPTIDE RETRO-INVERSO
GEVGNCPTSGAQYIETSIDRE
SEQ ID NO: 25 PK-2 (402-419) D PEPTIDE RETRO-INVERSO
AIKGTQGPAIQRVEDGRI
SEQ ID NO: 26 PK-3 (564-584) D PEPTIDE RETRO-INVERSO
IELTQPDRVADTTDAIDRGFQ
SEQ ID NO: 27 PK-4 (773-790) D PEPTIDE RETRO-INVERSO
KYIQKVQAFVEQTNKDQE
SEQ ID NO: 28 PK-S (1146-1163) D PEPTIDE RETRO-INVERSO
DPSTHNKFYKDLEEKFSD
SEQ ID NO: 29 PK-7 (485-502) D PEPTIDE RETRO-INVERSO
GNTPQFGYSQLPFYCNFG
SEQ ID NO: 30 PK-8 (369-392) D PEPTIDE RETRO-INVERSO
QRVEDGRIVESDAYVNTFCLDNLK
SEQ ID NO: 31 TT3-PK-1 (465-485) D PEPTIDE RETRO-INVERSO
FNNFTVSFWLRVPKVSASHLEGPSLGEVGNCPTSGAQYIETSIDRE
SEQ ID NO: 32 TT3-PK-2 (402-419) D PEPTIDE RETRO-INVERSO
FNNFTVSFWLRVPKVSASHLEGPSLAIKGTQGPAIQRVEDGRI
SEQ ID NO: 33 TT3-PK-3 (564-584) D PEPTIDE RETRO-INVERSO
FNNFTVSFWLRVPKVSASHLEGPSLIELTQPDRVADTTDAIDRGFQ
SEQ ID NO: 34 TT3-PK-4 (773-790) D PEPTIDE RETRO-INVERSO
FNNFTVSFWLRVPKVSASHLEGPSLKYIQKVQAFVEQTNKDQE
SEQ ID NO: 35 TT3-PK-5 (1146-1163) D PEPTIDE RETRO-INVERSO
FNNFTVSFWLRVPKVSASHLEGPSLDPSTHNKFYKDLEEKFSD
SEQ ID NO: 36 TT3-PK-7 (485-502) D PEPTIDE RETRO-INVERSO
FNNFTVSFWLRVPKVSASHLEGPSLGNTPQFGYSQLPFYCNFG
SEQ ID NO: 37 TT3-PK-8 (369-392) D PEPTIDE RETRO-INVERSO
FNNFTVSFWLRVPKVSASHLEGPSLQRVEDGRIVESDAYVNTFCLDNLK
SEQ ID NO: 38 PK-1.1 (465-485) with S477N substitution
ERDISTEIYQAGNTPCNGVEG
SEQ ID NO: 39 PK-1.2 (465-485) with S477N and E484K substitutions
ERDISTEIYQAGNTPCNGVKG
SEQ ID NO: 40 PK-1.3 (465-485) with S477N and E484Q substitutions
ERDISTEIYQAGNTPCNGVQG
SEQ ID NO: 41 PK-1.4 (465-485) with S477N, E484Q, and G485R substitutions
ERDISTEIYQAGNTPCNGVQR
SEQ ID NO: 42 PK-2.1 (402-419) with K417N substitution
IRGDEVRQIAPGQTGNIA
SEQ ID NO: 43 PK-7.1 (485-502) with F486L substitution
GLNCYFPLQSYGFQPTNG
SEQ ID NO: 44 PK-7.2 (485-502) with F490S substitution
GFNCYSPLQSYGFQPTNG
SEQ ID NO: 45 PK-7.3 (485-502) with S494P substitution
GFNCYFPLQPYGFQPTNG
SEQ ID NO: 46 PK-7.4 (485-502) with N501Y substitution
GFNCYFPLQSYGFQPTYG
SEQ ID NO: 47 PK-7.5 (485-502) with G485R substitution
RFNCYFPLQSYGFQPING
SEQ ID NO: 48 TT3-PK-1.1 (465-485) with S477N substitution
FNNFTVSFWLRVPKVSASHLEGPSLERDISTEIYQAGNTPCNGVEG
SEQ ID NO: 49 TT3-PK-1.2 (465-485) with S477N and E484K substitutions
FNNFTVSFWLRVPKVSASHLEGPSLERDISTEIYQAGNTPCNGVKG
SEQ ID NO: 50 TT3-PK-1.3 (465-485) with S477N and E484Q substitutions
FNNFTVSFWLRVPKVSASHLEGPSLERDISTEIYQAGNTPCNGVQG
SEQ ID NO: 51 TT3-PK-1.4 (465-485) with S477N, E484Q, and G485R substitutions
FNNFTVSFWLRVPKVSASHLEGPSLERDISTEIYQAGNTPCNGVQR
SEQ ID NO: 52 TT3-PK-2.1 (402-419) with K417N substitution
FNNFTVSFWLRVPKVSASHLEGPSLIRGDEVRQIAPGQTGNIA
SEQ ID NO: 53 TT3-PK-7.1 (485-502) with F486L substitution
FNNFTVSFWLRVPKVSASHLEGPSLGLNCYFPLQSYGFQPTNG
SEQ ID NO: 54 TT3-PK-7.2 (485-502) with F490S substitution
FNNFTVSFWLRVPKVSASHLEGPSLGFNCYSPLQSYGFQPTNG
SEQ ID NO: 55 TT3-PK-7.3 (485-502) with $494P substitution
FNNFTVSFWLRVPKVSASHLEGPSLGFNCYFPLQPYGFQPTNG
SEQ ID NO: 56 TT3-PK-7.4 (485-502) with N501Y substitution
FNNFTVSFWLRVPKVSASHLEGPSLGFNCYFPLQSYGFQPTYG
SEQ ID NO: 57 TT3-PK-7.5 (485-502) with G485R substitution
FNNFTVSFWLRVPKVSASHLEGPSLRFNCYFPLQSYGFQPTNG
SEQ ID NO: 58 internal control forward primer
CTCCCAACCCCAGAGGTAGT
SEQ ID NO: 59 internal control reverse primer
AGACCCCAGATCCAGAAAGG
SEQ ID NO: 60 hACE2: forward primer
CCTGGCTGAAAGACCAGAAC
SEQ ID NO: 61 hACE2: reverse primer
TCAAATTAGCCACTCGCACA
SEQ ID NO: 62 TT amino acid sequence
NSVDDALINSTIYSYFPSV
SEQ ID NO: 63 TT1 amino acid sequence
PGINGKAIHLVNNQSSE
SEQ ID NO: 64 P2 amino acid sequence
QYIKANSKFIGITEL
SEQ ID NO: 65 MVF (natural) amino acid sequence
LSEIKGVIVHRLEGV
SEQ ID NO: 66 HBV amino acid sequence
FFLLTRILTIPQSLN
SEQ ID NO: 67 CSP amino acid sequence
TCGVGVRVRSRVNAANKKPE
SEQ ID NO: 68 amino acid sequence spike protein SP|P59594|SPIKE_SARS
MFIFLLFLTLTSGSDLDRCTTFDDVQAPNYTQHTSSMRGVYYPDEIFRSDTLYLTQDLFL
PFYSNVTGFHTINHTFGNPVIPFKDGIYFAATEKSNVVRGWVFGSTMNNKSQSVIIINNST
NVVIRACNFELCDNPFFAVSKPMGTQTHTMIFDNAFNCTFEYISDAFSLDVSEKSGNFKH
LREFVFKNKDGFLYVYKGYQPIDVVRDLPSGFNTLKPIFKLPLGINITNFRAILTAFSPAQ
DIWGTSAAAYFVGYLKPTTFMLKYDENGTITDAVDCSONPLAELKCSVKSFEIDKGIYQ
TSNFRVVPSGDVVRFPNITNLCPFGEVENATKFPSVYAWERKKISNCVADYSVLYNSTFF
STFKCYGVSATKLNDLCFSNVYADSFVVKGDDVRQIAPGQTGVIADYNYKLPDDFMGC
VLAWNTRNIDATSTGNYNYKYRYLRHGKLRPFERDISNVPFSPDGKPCTPPALNCYWPL
NDYGFYTTTGIGYQPYRVVVLSFELLNAPATVCGPKLSTDLIKNQCVNFNFNGLTGTGV
LTPSSKRFQPFQQFGRDVSDFTDSVRDPKTSEILDISPCSFGGVSVITPGTNASSEVAVLY
QDVNCTDVSTAIHADQLTPAWRIYSTGNNVFQTQAGCLIGAEHVDTSYECDIPIGAGICA
SYHTVSLLRSTSQKSIVAYTMSLGADSSIAYSNNTIAIPTNFSISITTEVMPVSMAKTSVDC
NMYICGDSTECANLLLQYGSFCTQLNRALSGIAAEQDRNTREVFAQVKQMYKTPTLKY
FGGFNFSQILPDPLKPTKRSFIEDLLFNKVTLADAGFMKQYGECLGDINARDLICAQKFN
GLTVLPPLLTDDMIAAYTAALVSGTATAGWTFGAGAALQIPFAMQMAYRFNGIGVTQN
VLYENQKQIANQFNKAISQIQESLTTTSTALGKLQDVVNQNAQALNTLVKQLSSNFGAI
SSVLNDILSRLDKVEAEVQIDRLITGRLQSLQTYVTQQLIRAAEIRASANLAATKMSECV
LGQSKRVDFCGKGYHLMSFPQAAPHGVVFLHVTYVPSQERNFTTAPAICHEGKAYFPR
EGVFVFNGTSWFITQRNFFSPQIITTDNTFVSGNCDVVIGIINNTVYDPLQPELDSFKEELD
KYFKNHTSPDVDLGDISGINASVVNIQKEIDRLNEVAKNLNESLIDLQELGKYEQYIKWP
WYVWLGFIAGLIAIVMVTILLCCMTSCCSCLKGACSCGSCCKFDEDDSEPVLKGVKLHY
T
SEQ ID NO: 69 amino acid sequence spike protein SP|K9N5Q8|SPIKE_MERS1 (
MIHSVFLLMFLLTPTESYVDVGPDSVKSACIEVDIQQTFFDKTWPRPIDVSKADGIIYPQG
RTYSNITITYQGLFPYQGDHGDMYVYSAGHATGTTPQKLFVANYSQDVKQFANGFVVR
IGAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESG
VYSVSSFEAKPSGSVVEQAEGVECDFSPLLSGTPPQVYNFKRLVFTNCNYNLTKLLSLFS
VNDFTCSQISPAAIASNCYSSLILDYFSYPLSMKSDLSVSSAGPISQFNYKQSFSNPTCLIL
ATVPHNLTTITKPLKYSYINKCSRFLSDDRTEVPQLVNANQYSPCVSIVPSTVWEDGDYY
RKQLSPLEGGGWLVASGSTVAMTEQLQMGFGITVQYGTDINSVCPKLEFANDTKIASQ
LGNCVEYSLYGVSGRGVFQNCTAVGVRQQRFVYDAYQNLVGYYSDDGNYYCLRACV
SVPVSVIYDKETKTHATLFGSVACEHISSTMSQYSRSTRSMLKRRDSTYGPLQTPVGCVL
GLVNSSLFVEDCKLPLGQSLCALPDTPSTLTPRSVRSVPGEMRLASIAFNHPIQVDQLNSS
YFKLSIPTNFSFGVTQEYIQTTIQKVTVDCKQYVCNGFQKCEQLLREYGQFCSKINQALH
GANLRQDDSVRNLFASVKSSQSSPIIPGFGGDFNLTLLEPVSISTGSRSARSAIEDLLFDKV
TIADPGYMQGYDDCMQQGPASARDLICAQYVAGYKVLPPLMDVNMEAAYTSSLLGSI
AGVGWTAGLSSFAAIPFAQSIFYRLNGVGITQQVLSENQKLIANKFNQALGAMQTGFTT
TNEAFHKVODAVNNNAQALSKLASELSNTFGAISASIGDIIQRLDVLEQDAQIDRLINGR
LTTLNAFVAQQLVRSESAALSAQLAKDKVNECVKAQSKRSGFCGQGTHIVSFVVNAPN
GLYFMHVGYYPSNHIEVVSAYGLCDAANPTNCIAPVNGYFIKTNNTRIVDEWSYTGSSF
YAPEPITSLNTKYVAPQVTYQNISTNLPPPLLGNSTGIDFQDELDEFFKNVSTSIPNFGSLT
QINTTLLDLTYEMLSLQQVVKALNESYIDLKELGNYTYYNKWPWYIWLGFIAGLVALA
LCVFFILCCTGCGTNCMGKLKCNRCCDRYEEYDLEPHKVHVH
SEQ ID NO: 70 amino acid sequence COV1
ERDISNVPFSPDGKPCTPPA
SEQ ID NO: 71 amino acid sequence MERS
TEVPQLVNANQYSPCVSIVP
SEQ ID NO: 72 amino acid sequence COV1
VKGDDVRQIAPGQTGVIA
SEQ ID NO: 73 amino acid sequence MERS
YPLSMKSDLSVSSAGPIS
SEQ ID NO: 74 amino acid sequence COV1
QFGRDVSDFTDSVRDPKTSEI
SEQ ID NO: 75 amino acid sequence MERS
RFVYDAYQNLVGYYSDDGNY
SEQ ID NO: 76 amino acid sequence COV1
EQDRNTREVFAQVKQMYK
SEQ ID NO: 77 amino acid sequence MERS
RQDDSVRNLFASVKSSQS
SEQ ID NO: 78 amino acid sequence COV1
DSFKEELDKYFKNHTSPD
SEQ ID NO: 79 amino acid sequence MERS
IDFQDELDEFFKNVSTSI
SEQ ID NO: 80 amino acid sequence COV1
ALNCYWPLNDYGFYTTTG
SEQ ID NO: 81 amino acid sequence MERS
PSTVWEDGDYYRKQLSPLEGGGWLVASG
SEQ ID NO: 82 amino acid sequence COV1
YNSTFFSTFKCYGVSATKLNDLCF
SEQ ID NO: 83 amino acid sequence MERS
LSLFSVNDFTCSQISPAAIASNCY

### F. References

Beigel J H, Tomashek K M, Dodd L E, Mehta A K, Zingman B S, Kalil A C: Remdesivir for the Treatment of Covid-19—Final Report. 2020, 383(19):1813-1826.
Dakappagari N K, Douglas D B, Triozzi P L, Stevens V C, Kaumaya P T: Prevention of mammary tumors with a chimeric HER-2 B-cell epitope peptide vaccine. Cancer Res 2000, 60(14):3782-3789.
Foy K C, Miller M J, Moldovan N, Carson W E, 3rd, Kaumaya P T: Combined vaccination with HER-2 peptide followed by therapy with VEGF peptide mimics exerts effective anti-tumor and anti-angiogenic effects in vitro and in vivo. Oncoimmunology 2012, 1(7):1048-1060.
Grein J, Ohmagari N, Shin D, Diaz G, Asperges E, Castagna A, Feldt T, Green G, Green M L, Lescure F X et at: Compassionate Use of Remdesivir for Patients with Severe Covid-19. 2020, 382(24):2327-2336.
Haddar C, Verhoeven P O, Bourlet T, Pozzetto B, Pillet S: Brief comparative evaluation of six open one-step R T-qPCR mastermixes for the detection of SARS-CoV-2 RNA using a Taqman probe. Journal of clinical virology: the official publication of the Pan American Society for Clinical Virology 2020, 132:104636.
Kalil A C, Patterson T F, Mehta A K, Tomashek K M, Wolfe C R, Ghazaryan V, Marconi V C, Ruiz-Palacios G M, Hsieh L, Kline S et al: Baricitinib plus Remdesivir for Hospitalized Adults with Covid-19. 2021, 384(9):795-807.
Kaumaya P T: A paradigm shift: Cancer therapy with peptide-based B-cell epitopes and peptide immunotherapeutics targeting multiple solid tumor types: Emerging concepts and validation of combination immunotherapy. Hum Vaccin Immunother 2015, 11(6):1368-1386.
Kaumaya PTP, Kobs-Conrad S, DiGeorge A M, Stevens V: Denovo Engineering of Protein Immunogenic & Antigenic Determinants. In: PEPTIDES. Edited by Anantharamaiah G M B, C., vol. 9: Springer-Verlag.; 1994: 133-164.
McCray P B, Jr., Pewe L, Wohlford-Lenane C, Hickey M, Manzel L, Shi L, Netland J, Jia H P, Halabi C, Sigmund C D et al: Lethal infection of K18-hACE2 mice infected with severe acute respiratory syndrome coronavirus. Journal of virology 2007, 81(2):813-821.
Menachery V D, Yount B L, Jr., Sims A C, Debbink K, Agnihothram S S, Gralinski L E, Graham R L, Scobey T, Plante J A, Royal S R et al: SARS-like WIV1-CoV poised for human emergence. Proceedings of the National Academy of Sciences of the United States of America 2016, 113(11):3048-3053.
Netland J, Meyerholz D K, Moore S, Cassell M, Perlman S: Severe acute respiratory syndrome coronavirus infection causes neuronal death in the absence of encephalitis in mice transgenic for human ACE2. Journal of virology 2008, 82(15):7264-7275.
Pravin T. P Kaumaya, Jay Overholser, LinLin Guo, Manuel Penichet and Tanios Bekaii-Saab. Immunogenicity and antitumor efficacy of a novel human PD-1 B-cell vaccine (PD1-Vaxx) and synergistic combination immunotherapy with dual trastuzumab/pertuzumab HER-2 B-cell epitope vaccines (B-Vaxx) in syngeneic mouse models. OncoImmunology 2020, Vol. 9, No. 1, e1818437
Pravin T. P Kaumaya. B-cell epitope peptide cancer vaccines: A new paradigm for combination immunotherapies with novel checkpoint peptide vaccine. Future Oncology (2020) 16(23), 1767-1791
Tseng C T, Huang C, Newman P, Wang N, Narayanan K, Watts D M, Makino S, Packard M M, Zaki S R, Chan T S et al: Severe acute respiratory syndrome coronavirus infection of mice transgenic for the human Angiotensin-converting enzyme 2 virus receptor. Journal of virology 2007, 81(3):1162-1173.
V'Kovski P, Kratzel A, Steiner S, Stalder H: Coronavirus biology and replication: implications for SARS-CoV-2. 2021, 19(3):155-170.
Zheng J, Wong L R: COVID-19 treatments and pathogenesis including anosmia in K18-hACE2 mice. 2021, 589(7843):603-607.

1. A chimeric peptide for stimulating an immune response to a coronavirus spike protein comprising one or more SARS-CoV-2 B cell epitopes, a T helper (Th) epitope, and a linker joining the SARS-CoV-2 B cell epitope to the Th epitope, wherein the one or more SARS-CoV-2 B cell epitopes consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

2. The chimeric peptide of claim 1, wherein the amino acids comprising the SARS-CoV-2 B cell epitope are the D enantiomer.

3. The coronavirus spike protein synthetic peptide of claim 1, wherein the SARS-CoV-2 B cell epitope is acetylated.

4. The chimeric peptide of claim 1, wherein the Th epitope comprises a measles virus fusion protein peptide.

5. The chimeric peptide of claim 1, wherein the Th epitope comprises SEQ ID NO: 11.

6. The chimeric peptide of claim 1, wherein the Th epitope comprises a tetanus toxoid(TT3) peptide.

7. The chimeric peptide of claim 1, wherein the Th epitope comprises SEQ ID NO: 12.

8. The chimeric peptide of claim 1, wherein the linker comprises SEQ ID NO: 13.

9. The chimeric peptide of claim 1, wherein the peptide comprises the amino acid sequence as set forth in SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and/or SEQ ID NO: 23.

10. A coronavirus spike protein synthetic peptide for stimulating an immune response to a coronavirus spike protein comprising one or more of the sequences as set forth in SEQ ID NO: 24, SEQ ID NO: 25, SEQ ID NO: 26, SEQ ID NO: 27, SEQ ID NO: 28, SEQ ID NO: 29, or SEQ ID NO: 30.

11. (canceled)

12. (canceled)

13. (canceled)

14. (canceled)

15. (canceled)

16. (canceled)

17. (canceled)

18. (canceled)

19. The chimeric peptide of claim 10, wherein the coronavirus spike protein synthetic peptide comprises the amino acid sequence as set forth in SEQ ID NO: 31, SEQ ID NO: 32, SEQ ID NO: 33, SEQ ID NO: 34, SEQ ID NO: 35, SEQ ID NO: 36, or SEQ ID NO: 37.

20. A modified coronavirus spike protein peptide for stimulating an immune response to a coronavirus spike protein comprising one or more of the sequences as set forth in SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47.

21. (canceled)

22. (canceled)

23. (canceled)

24. (canceled)

25. (canceled)

26. (canceled)

27. (canceled)

28. (canceled)

29. The chimeric peptide of claim 20, wherein the modified coronavirus spike protein peptide comprises the amino acid sequence as set forth in SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57.

30. (canceled)

31. (canceled)

32. (canceled)

33. (canceled)

34. (canceled)

35. A method of treating a coronavirus infection in a subject comprising administering to the subject any of the peptides, antibodies, or compositions of claim 1.

36. A method of treating a coronavirus infection in a subject comprising administering to the subject one or more chimeric peptides comprising a SARS-CoV-2 B cell epitope, a T helper (Th) epitope, and a linker joining the SARS-CoV-2 B cell epitope to the Th epitope, wherein the SARS-CoV-2 B cell epitope consist of a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

37. The method of treating a coronavirus infection of claim 36, wherein the one or more chimeric peptides comprises SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and/or SEQ ID NO: 23.

38. A method of treating a coronavirus infection in a subject comprising i) administering to the subject one or more coronavirus spike protein synthetic peptides wherein the coronavirus spike protein synthetic peptide comprises one or more of the sequences as set forth in SEQ ID NO: 24, SEQ ID NO: 25, SEQ ID NO: 26, SEQ ID NO: 28, SEQ ID NO: 29, or SEQ ID NO: 30; or ii) administering one or more chimeric synthetic peptides comprising a coronavirus spike protein synthetic peptides, a T helper (Th) epitope, and a linker joining the coronavirus spike protein synthetic peptides to the Th epitope; wherein the coronavirus spike protein synthetic peptide comprises SEQ ID NO: 24, SEQ ID NO: 25, SEQ ID NO: 26, SEQ ID NO: 28, SEQ ID NO: 29, or SEQ ID NO: 30.

39. The method of treating a coronavirus infection of claim 38, wherein the one or more chimeric peptides comprises SEQ ID NO: 31, SEQ ID NO: 32, SEQ ID NO: 33, SEQ ID NO: 34, SEQ ID NO: 35, SEQ ID NO: 36, or SEQ ID NO: 37.

40. A method of treating a coronavirus infection in a subject comprising
i) administering to the subject one or more modified coronavirus spike protein peptides wherein the coronavirus spike protein synthetic peptide comprises one or more of the sequences as set forth in SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47; or
ii) administering one or more chimeric synthetic peptides comprising a modified coronavirus spike protein peptides, a T helper (Th) epitope, and a linker joining the coronavirus spike protein synthetic peptides to the Th epitope; wherein the modified coronavirus spike protein peptide comprises SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and/or SEQ ID NO: 47.

41. The method of treating a coronavirus infection of claim 40, wherein the one or more chimeric peptides comprises SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and/or SEQ ID NO: 57.

42. (canceled)
